(19) United States
(12) Patent Application Publication
(10) Pub. No.: US 2024/0242835 A1
(43) Pub. Date: Jul. 18, 2024

(54) AUTOMATED DIGITAL ASSESSMENT OF HISTOLOGIC SAMPLES

(71) Applicant: Genentech, Inc., South San Francisco, CA (US)

(72) Inventors: Jennifer Margaret GILTNANE, Hillsborough, CA (US); Katja SCHULZE, Greenbrae, CA (US)

(21) Appl. No.: 18/412,348

(22) Filed: Jan. 12, 2024

Related U.S. Application Data
(63) Continuation of application No. PCT/US22/37385, filed on Jul. 15, 2022.
(60) Provisional application No. 63/222,413, filed on Jul. 15, 2021.
(60) Provisional application No. 63/254,992, filed on Oct. 12, 2021.
(60) Provisional application No. 63/314,984, filed on Feb. 28, 2022.

(51) Int. Cl. G16H 50/20; G06T 7/00; G06T 7/11

(52) U.S. Cl. CPC G16H 50/20; G06T 7/0014; G06T 7/11; G06T 2200/24; G06T 2207/20081; G06T 2207/20092; G06T 2207/30096

(57) ABSTRACT

In one embodiment, a digital pathology image processing system receives digital pathology images of histologic samples. Physical characteristics of a first histologic sample associated with a first digital pathology image are assessed. The first digital pathology image is segmented based on one or more regions of the first digital pathology image corresponding to tumor bed. The first digital pathology image is also segmented based on one or more regions corresponding to one or more predetermined histologic features. An assessment is generated regarding a specified condition in the first histologic sample based on the one or more regions corresponding to tumor bed and the one or more regions corresponding to the one or more predetermined histologic features. The condition may be associated with a level or degree of pathologic response. A user interface may display information related to the assessment.

### PRIORITY

This application claims the benefit, under 35 U.S.C. § 119(e), of U.S. Provisional Patent Application No. 63/222,413, filed 15 Jul. 2021, U.S. Provisional Patent Application No. 63/254,992, filed 12 Oct. 2021, and U.S. Provisional Patent Application No. 63/314,984, filed 28 Feb. 2022, which are incorporated herein by reference.

### TECHNICAL FIELD

This disclosure generally relates to tools for assessing responses of tumors to selected stimuli and evaluating the response thereto, including treatment effects.

### BACKGROUND

Current guidance provided by International Association for the Study of Lung Cancer (IASLC) presents recommendations on how to process and evaluate resected lung cancer specimens after neoadjuvant therapy in the setting of clinical trials and clinical practice, as well as how to assess treatment effects in biopsies obtained during neoadjuvant therapy (prior to surgical resection). However, standardization and harmonized implementation of this guidance into clinical practice and clinical trial sites is limited. Suggestions have been provided on definitions for various levels or degrees of pathologic response, including major pathologic response (MPR) or pathologic complete response (pCR), and recommendations for the manual processing and evaluation of lung cancer resection specimens have been made, including initial identification of definitions for pathologic response. Pathologic response, including pathologic complete response (pCR) and major pathologic response (MPR) is a histologic assessment providing an early measure of treatment efficacy MPR and pCR have been studied as a surrogate for disease-free survival (DFS), event-free survival (EFS), relapse-free survival (RFS) or overall survival (OS) and have been used as an efficacy endpoint in Phase II and Ill clinical trials studying neoadjuvant therapies in resectable non-small cell lung cancer (NSCLC) and breast cancer.

It has been proposed that these definitions can be applied to all systemic therapies, including chemotherapy, chemoradiation, molecular-targeted therapy, immunotherapy, or any future novel therapies yet to be discovered, whether administered alone or in combination. Assessment of pathologic response and treatment effects could be used to determine the quality and quantity of biological changes (e.g., fibrosis, necrosis), to identify qualitative/descriptive features in response to therapy, to provide prognostic information, or to risk-stratify patients for subsequent post-surgical patient management (e.g., adjuvant therapy). Standard pathologic response assessment is expected to allow for correlations with EFS, DFS, RFS and OS in ongoing and future trials. However, reliable correlation of MPR with DFS, EFS, and OS has been hampered by lack of large studies. In particular, there is a need for a surrogate endpoint because EFS, DFS, and OS take a long time to read out and are influenced by adjuvant therapy and other biases. Pathologic response assessment provides an early meaningful readout after neoadjuvant therapy. Furthermore, the rubric for pathology scoring of tumor specimens may differ for treatments such as immunotherapy that may impact the tumor microenvironment and its response to therapy differently from other modalities. However, current specimen processing is subjective, inconsistent, labor intensive, and not scalable, and it is largely performed at large academic centers, or by specialized thoracic pathologists. A desire exists to standardize and scale specimen evaluations and improve reliability of the evaluations.

### SUMMARY OF PARTICULAR EMBODIMENTS

In particular embodiments, a one or more computer-readable non-transitory storage media includes, receiving, by a digital pathology image processing system, a plurality of digital pathology images of histologic samples. The digital pathology image processing system assesses physical characteristics of a first histologic sample associated with a first digital pathology image of the plurality of digital pathology images. The digital pathology image processing system segments the first digital pathology image based on one or more regions of the first digital pathology image corresponding to tumor bed. The digital pathology image processing system segments the first digital pathology image based on one or more regions of the first digital pathology image corresponding to one or more predetermined histologic features. The digital pathology image processing system generates an assessment regarding a specified condition in the first histologic sample based on the one or more regions of the first digital pathology image corresponding to tumor bed and the one or more regions of the first digital pathology image corresponding to the one or more predetermined histologic features. The digital pathology image processing system generates a user interface including a display of the assessment.

In particular embodiments, the digital pathology image processing system segments the first digital pathology image using a machine-learning model trained to segment tumor bed regions separately from non-tumor bed regions. In particular embodiments, the digital pathology image processing system segments the first digital pathology image using a machine-learning model trained to segment the one or more predetermined histologic features from tumor bed regions. In particular embodiments, the digital pathology image processing system receives feedback from a user operator regarding the assessment and re-trains the machine-learning model based on the feedback. In particular embodiments, the one or more predetermined histologic features comprise necrotic tumor cells, regions of necrosis, viable tumor cells, regions of viable tumor, tumor stroma cells, or regions of tumor stroma. In particular embodiments, generating the assessment regarding the specified condition includes determining whether the specified condition is present.

In particular embodiments, the digital pathology image processing system computes a first area value of the first histologic sample corresponding to tumor bed based on the one or more regions of the first digital pathology image corresponding to tumor bed and the physical characteristics of the first histologic sample. The digital pathology image processing system computes a second area value of the first histologic sample corresponding to each of the one or more predetermined histologic features based on the one or more regions of the first digital pathology image corresponding to the one or more predetermined histologic features and the physical characteristics of the first histologic sample. The assessment regarding the specified condition in the first histologic sample is generated based on the first area value and the second area value. In further embodiments, determining whether the specified condition is detected in the first histologic sample based on the first area value and the second area value includes computing a percentage of the second area relative to the first area corresponding to each of the one or more predetermined histologic features. In further embodiments, determining whether the specified condition is detected in the first histologic sample based on the first area value and the second area value includes determining whether the percentage of the second area value relative to the first area value satisfies one or more predetermined thresholds, wherein the one or more predetermined thresholds are based on the specified condition. In particular embodiments, segmenting the first digital pathology image based on the one or more regions of the first digital pathology image corresponding to tumor bed includes producing a first instance of the first digital pathology image including annotations corresponding to the regions of the first digital pathology image corresponding to tumor bed and segmenting the first digital pathology image based on the one or more regions of the first digital pathology image corresponding to the one or more predetermined histologic features includes producing a second instance of the first digital pathology image including annotations corresponding to the regions of the first digital pathology image corresponding to the one or more predetermined histologic features.

In particular embodiments, the first histologic sample is further associated with a set of one or more second digital pathology images. For each of the set of second digital pathology images the digital pathology image processing system assesses physical characteristics of the first histologic sample associated with the second digital pathology image, segments the second digital pathology image based on one or more regions of the second digital pathology image corresponding to tumor bed, and segments the second digital pathology image based on one or more regions of the second digital pathology image corresponding to the one or more predetermined histologic features. Generating the assessment regarding the specified condition in the first histologic sample is further based on the one or more regions of the set of second digital pathology images corresponding to tumor bed and the one or more regions of the set of second digital pathology images corresponding to the one or more predetermined histologic features. In particular embodiments, the digital pathology image processing system receives a human-generated assessment of the first histologic sample. The digital pathology image processing system compares the assessment generated by the first digital pathology image processing system to the human-generated assessment. The digital pathology image processing system generates a user interface including a display of the comparison. In particular embodiments, the assessment regarding the specified condition is further generated based on metadata and additional data associated with the first histologic sample. In particular embodiments, the digital pathology image processing system generates a level of confidence in the assessment. In particular embodiments, the user interface including the display of the assessment further includes a display of annotations for the first digital pathology image associated with the segmentations based on the one or more regions of the first digital pathology image corresponding to tumor bed and the one or more regions of the first digital pathology image corresponding to the one or more predetermined histologic features. In particular embodiments, the specified condition corresponds to a level or degree of pathologic response.

The embodiments disclosed herein are only examples, and the scope of this disclosure is not limited to them. Particular embodiments may include all, some, or none of the components, elements, features, functions, operations, or steps of the embodiments disclosed herein. Embodiments according to the invention are in particular disclosed in the attached claims directed to a method, a storage medium, a system and a computer program product, wherein any feature mentioned in one claim category, e.g., method, may be claimed in another claim category, e.g., system, as well. The dependencies or references back in the attached claims are chosen for formal reasons only. However any subject matter resulting from a deliberate reference back to any previous claims (in particular multiple dependencies) may be claimed as well, so that any combination of claims and the features thereof are disclosed and may be claimed regardless of the dependencies chosen in the attached claims. The subject-matter which may be claimed includes not only the combinations of features as set out in the attached claims but also any other combination of features in the claims, wherein each feature mentioned in the claims may be combined with any other feature or combination of other features in the claims. Furthermore, any of the embodiments and features described or depicted herein may be claimed in a separate claim and/or in any combination with any embodiment or feature described or depicted herein or with any of the features of the attached claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a network of interacting computer systems that may be used, as described herein according to some embodiments of the present disclosure.

FIG. 2 illustrates an example method for automated model-based assessment of pathologic response.

FIG. 3 illustrates an example process for automated assessment of major pathologic response using machine-learning models.

FIG. 4 illustrates an example process for training models for a tool for automated assessment of pathologic response.

FIGS. 5A and 5B illustrate an example workflow integration of a model-based MPR assessment tool.

FIGS. 6A-8B illustrate examples of digital pathology images and annotation outputs of a pathologic response assessment tool.

FIGS. 9-11 illustrate example user interfaces of a pathologic response assessment tool.

FIG. 12A illustrates a manual workflow for clinicians evaluating histology samples. FIG. 12B illustrates a digital workflow for automated assessment of pathologic response.

FIGS. 13A-13D illustrate various approaches to comparing manual assessment of a percentage of the tumor bed exhibiting viable tumor as between a local pathologist and three central reviewers.

FIGS. 14A-14B illustrate various approaches to assessing performance of digital assessment of a percentage of the tumor bed exhibiting viable tumor by way of comparison to results obtained through manual assessment.

FIGS. 15A-15B illustrate whole slide images depicting small regions of viable tumor.

FIG. 16 shows two graphs comparing correlations and discrepancies between manual assessment of pathologic response and digital assessment of pathologic response.

FIGS. 17A-17D illustrate four graphs showing differences between digitally assessed MPR versus manually assessed MPR with respect to DFS and OS.

FIG. 18 illustrates an example computer system.

### DESCRIPTION OF EXAMPLE EMBODIMENTS

Standard treatment options for patients with resectable lung cancer have been settled for more than two decades. As an example, preoperative chemotherapy became a standard option when a meta-analysis of neoadjuvant chemotherapy trials reported that preoperative platinum-doublet chemotherapy improved survival over operation alone in resectable early-stage NSCLC. The magnitude of improvement of survival outcome is thought to be significant with manageable hazard ratios. Although large and lengthy adjuvant studies assessing new strategies beyond chemotherapy have to date yielded negative results, systemic therapy for advanced lung cancer has evolved such that for certain subgroups of patients, platinum-doublet chemotherapy alone has become less common in comparison to other options such as, for example, the use of tyrosine kinase inhibitors, immunotherapy alone, and chemotherapy plus checkpoint inhibitors. Conversely, there have been no changes to the standard of care in resectable disease in close to twenty years. Studies are needed to move these advances into the curative setting, which remains a highly unmet need with only a 5% five-year survival benefit.

In neoadjuvant trials, the efficacy of the therapy cannot be determined until years later when EFS, DFS, RFS, and OS are available. These endpoints are therefore blunt tools with high potential for conflating factors and requiring considerable investment to track results over time. This may delay the time before definitive results can be determined as well as the overall value of the results. Neoadjuvant trials allow efficacy end points such as clinical and pathologic response to be determined in several months. Neoadjuvant treatments offer potential advantages, including the ability to treat micrometastatic disease and analyze the treatment-related effect on the primary tumor after or during either adjuvant or neoadjuvant therapy. Neoadjuvant treatment with surrogate measures of efficacy (e.g., surrogate endpoints) such as pathologic response or histologic treatment effect have the potential to accelerate curative therapies for this patient population. Determining response to neoadjuvant therapy may be useful not only as a surrogate endpoint, but also to differentiate the neoadjuvant treatment effect from the adjuvant treatment effect if patients receive both types of treatment during a clinical trial or in practice. It could provide potential prognostic information at time of surgery, thereby elucidating treatment effects from neoadjuvant therapy which may help to inform better adjuvant treatment strategies. And from a patient perspective, such measures of efficacy may be able to provide support for the value of such therapy, thereby supporting an argument for support of such therapy by healthcare providers and insurers.

Currently, there is limited established guidance on how to process and evaluate resected lung cancer specimens after neoadjuvant therapy in the setting of clinical trials and clinical practice. There is also a lack of precise definitions for certain levels or degrees of pathologic response, including major pathologic response (MPR) or pathologic complete response (pCR). For the purposes of this disclosure, MPR may refer to the reduction of residual viable tumor to the amount beneath an established clinically significant cutoff based on prior evidence according to the individual histologic type of lung cancer or other cancers and a specific therapy. PCR may refer to a lack of any residual viable tumor on review of hematoxylin and eosin (H&E)-stained slides after complete evaluation of a resected lung cancer specimen including all sampled regional lymph nodes. Data from retrospective and/or non-randomized single-arm studies have indicated that patients, particularly with lung cancers, that show an MPR may be defined as 10% or less residual viable tumor after neoadjuvant therapy may have a significantly improved survival rate. This reporting has led to the design of neoadjuvant therapy clinical trials for resectable lung cancer in which MPR is a primary or co-primary endpoint due to being a quantifiable measure for evaluating results by evaluating resected samples directly MPR or pCR assessment is currently performed manually by pathologists. In addition to being labor intensive and not scalable, the assessments often lack consistency between individual pathologists and among assessments by the same pathologist over time. In certain types of cancers in the clinical setting and elsewhere, that can be used to enforce and further facilitate the goals of consistency of evaluations and improve the overall collection of pathologic data. Moreover, there is a lack of automated tools to replace the subjective and labor intensive practices required to assess MPR or pCR at a scalable level.

There is a noted lack of standardization for the calculation of metrics associated with histologic features of interest and assessment of MPR among potentially widely varying clinical studies and even among pathologists within an individual study. In many cases, the determination of what constitutes a histologic feature or cell type of interest for assessment of MPR, as discussed herein, can be a highly subjective practice and hindered by co-existent pathologies or lack of experience in the practice. While eventual alignment across multiple pathologists is expected, the time-consuming and arduous manual process for inspecting slides or digital pathology images of the slides reduces the opportunity for reviewing pathologists to efficiently check the work of other pathologists and enforce consistency of results. There is no widespread adoption of a straightforward standardized tool for assessment of MPR. Each pathologist or study technician may have their own standards for using particular sample slides, scoring individual samples, and potentially weighting the contribution of each measured sample to an overall assessment of MPR for the purpose of declaring clinical results.

To provide much-needed consistency and reliability in pathologic response assessment, the embodiments herein provide computer-implemented tools and methods to be used in assessing MPR or pCR. The present embodiments may be used to assess MPR or pCR for certain types of cancers in the clinical setting and in real-world settings that can be used to enforce and further facilitate the goals of consistency of evaluations. A systematic assessment may help extract data for real-world databases or enable retrospective real-world data studies of higher quality. Furthermore, the present embodiments may improve the overall collection of pathologic response data and allow for integration with other standardized variables such as R status, surgical outcomes, RECIST response, blood-based biomarkers, liquid biopsy, and downstaging to better characterize a response to therapy and inform subsequent management of a patient. The systems and methods described herein may replace the subjective and labor intensive practices required to assess MPR or pCR at a scalable level. Described herein are techniques for the automated evaluation of pathologic response using one or more trained machine-learning models to detect the cell types-of-interest and regions-of-interest of a sample slide. There is a need for a tool to automatically calculate metrics associated with histologic features of interest and provide assessment of MPR, pCR, or other pathologic response evaluations, as well as quantification and assessment of other types of treatment effects and differentiate from other reactive patterns. The present disclosure provides embodiments of such a tool that automatically calculates of metrics associated with histologic features of interest, assesses pathologic response (including MPR and pCR), and quantifies/assesses treatment effects or reactive patterns, based on a provided digital pathology image of a slide, which could be derived from one or more surgical resection specimens or biopsy specimens.

In particular embodiments, one or more machine-learning models are trained to evaluate digital pathology images for digital pathologic assessment. In one instance, a system may comprise two or more co-trained models. A first model may be trained to determine portions of an image that comprise a tumor bed and a portion of the image that comprise other areas. The tumor bed may be defined as an area where the original (pre-treatment) tumor is considered to have been located. The tumor bed may include residual viable tumor cells, along with concurrent necrosis (necrotic tumor cells) and tumor stroma cells (which may include, for example, vascular cells, fibroblasts, mesenchymal stromal cells, or inflammatory cells). The area identified as tumor bed area may exclude cholesterol clefts, reactive tissue unrelated to cancer (e.g., inflammatory cells that are part of the reactive changes surrounding the tumor bed), other non-cancer associated pathologic changes (e.g., histoplasmosis), or other technical artifacts that may cause an area to be un-analyzable (e.g., folded tissue, pen marks, air bubbles). The first model may be trained to receive a digital pathology image, such as a digitized H&E-stained image, and annotate or otherwise indicate which regions of the image include the tumor bed and which regions of the image do not. The first model may also be able to detect tumor bed regions in lymph nodes. A second model may be trained to determine cell types- and regions-of-interest or histologic features based on the condition being assessed. As described herein, in the case of digital pathologic response assessment for lung cancers, the cell types-of-interest and regions-of-interest may comprise viable tumor cells, regions of viable tumor, necrotic tumor cells, regions of necrosis, tumor stroma cells, or regions of tumor stroma. The second model may be trained to receive the same digital pathology image and annotate or otherwise indicate which regions of the image include viable tumor, tumor stroma, and necrosis. The second model may further determine the total area within the digital pathology image corresponding to each type of cell within the tumor bed (e.g., the area of the tumor bed that comprises viable tumor cells, the area of the tumor bed that comprises tumor stroma cells, and the area of the tumor bed that comprises necrotic tumor cells). As discussed, these cells contribute to the tumor bed, so the total area of the determined regions is expected to entail the area of the tumor bed. The relative area of each region may be calculated as the area of the region divided by the total area of the tumor bed.

The automated evaluation of pathologic response based on received digital pathology images involves several steps. In particular embodiments, evaluating the sample includes taking measurements of the profile of the sample, such as the size, shape, weight, density, etc. These measurements may be performed, during the preparation of the sample, by or using the components of the digital pathology image generation system discussed herein. Then, based on the type of histologic feature being evaluated, presenting criteria for the histologic feature are evaluated by machine-learning models. First, regions of the digital pathology image are identified as being relevant to the assessment of pathologic response. More particularly, the machine-learning models are trained to identify cell types-of interest, cell morphology, and/or regions-of-interest in order to identify distinctions, such as, by way of example and not limitation, tumor bed or not, viable tumor cells or necrotic tumor cells, tumor cells or tumor stroma cells, or region of viable tumor or region of tumor stroma. This assessment may also define a sample area under analysis. As used herein, the term “sample area” may be used to refer to any suitable component or subdivision of a sample under analysis, for example a suitable sample area may be dependent on the type of analysis being performed. As an example only, and not by way of limitation, sample area may be used interchangeably or in conjunction with some or all of a slide, tumor bed, sample margins, sample cassette, or other suitable method of carrying and recording samples of interest.

The regions that are assessed as being relevant to the assessment of pathologic response may be measured or quantified. As an example, digital image analysis tools may be used with supplied metadata regarding how the digital pathology images are generated to determine the area of each of the cell types- and/or regions-of-interest. Example metadata may include the magnification used to capture the digital pathology image, the pixel density of the image and the image sensor, the type of scanner used, etc. The machine-learning model may also be trained to determine other characteristics, such as an approximate oxygenation level of the histologic feature or a degree of exposure or production of various components. Once the area of the various cell types- and/or regions-of-interest are assessed, the machine learning model calculates the percentage of the sample area that comprises or exhibits certain types of cells or regions.

Measurements or quantifications of the assessed regions may be compared. For example, the size of the region corresponding to each of the viable tumor, tumor stroma, and necrotic tumor is compared to the tumor bed. The resulting values, which may be used in the form of percentages are then compared to one or more predetermined threshold values. The predetermined threshold values may have been determined based on best practices or prior studies as being indicative of certain types of response. For example, a tumor cell area percentage of less than 10% may be indicative of MPR. A tumor cell area percentage of less than 1% may be indicative of pCR. The resulting assessment is then output to a user. This pathologic response data may be used to identify a cut-off for digital response assessment that is prognostic or predictive with correlation to DFS, OS, RFS, or EFS, risk stratification, or patient selection, which could be specific to a histologic or molecular sub-type or a specific treatment regimen used.

In particular embodiments, MPR may be assessed based on the relative amount of a tumor or other defined mass of interest contained in a given unit of a sample. As an example, a MPR may be assessed for a tumor based on an individual slide or sample of the tumor. The accuracy of the MPR assessment may be enhanced by using a larger number of measurements, for example using multiple slides for a given block of a sample. In particular, MPR may be assessed based on the amount of the tumor that can be classified as exhibiting certain conditions based on the assessment of the conditions by, for example, a pathologist. For example, the MPR assessment may be based on the number of viable cells in a tumor bed, wherein the tumor bed also includes regions of necrosis and stroma (which may include inflammation). In such cases, the presence and documentation of necrosis and stroma in the tumor bed is useful to describe and distinguish complex response profiles. The areas occupied by each type of predetermined histologic feature detected in the tumor bed may be expressed in terms of percentages, so that the total must equal to 100%. Thus, in the case of a sample of a tumor, the area of the tumor may be divided into a percentage of the tumor bed that exhibits or includes viable tumor, a percentage of the tumor bed that exhibits or includes necrosis, and a percentage of the tumor bed that exhibits or includes the tumor stroma.

From the above described percentages for each sample, an average percentage of each type of histologic feature may be collected across the slides under analysis for a given block to determine an approximate amount of each across the block (e.g., across the resected sample). For example, where the histologic features of interest include viable tumor cells, necrosis, and tumor stroma, an average percentage of regions of viable tumor, necrosis, and tumor stroma may each be calculated across the block.

In certain embodiments, MPR may be evaluated by comparing the computed average value of one or more of these histologic features to a predetermined threshold. The predetermined threshold may be based, for example, on the indication sought, the type of mass being evaluated, a category of the disease being evaluated, etc. For example, a block of resected sample of the tumor may be said to exhibit MPR where the average percentage of the block comprising viable tumor is below 10%. As this average is calculated across the number of slides, but necessarily relative to the surface area of each individual slide, this calculated number may be referred to as a non-weighted average.

Additionally, or alternatively, a weighted average may be calculated using the area of each sample, such that a more accurate calculation for the percentage of the block comprising or exhibiting certain conditions can be evaluated. In this way, larger samples will contribute more to the average than smaller samples. In addition to the area of the sample, other metrics to account for the value of information provided by each slide may be used, such as by evaluating the mass of a slide relative to the block, the density of the slide relative to the block, the position in the block from which the material in the slide was taken, or other objective measures. As an example, in the case of using the area of the slide to weight the contribution of each slide to the evaluation of the block, the percentage of the block exhibiting a particular condition may be calculated as

$${\sum }_{i}^{n} \frac{{l}_{i} {w}_{i}}{{A}_{b}} {C}_{i}$$

where n is the number of samples, lᵢ is the length (e.g., in cm) of the material under evaluation in slide i, wᵢ is the width (e.g., in cm) of the material under evaluation in slide i, A_{b}, is the total area (e.g., in cm²) of all slides in block b of the sample (i.e., A_{b}=Σᵢ₌₁ⁿlᵢwᵢ), and Cᵢ is value of the percentage of slide i comprising the histologic feature of interest for the slide i. As an example, the weighted percentage of a block comprising viable tumor may be calculated as

$${\sum }_{i}^{n} \frac{{l}_{i} {w}_{i}}{{A}_{b}} {V}_{i}$$

where Vᵢ is the percentage of slide i comprising viable tumor. In particular embodiments, it may be beneficial to compute and compare the weighted and non-weighted averages of the histologic features of interest as an efficient comparison between clinically validated response mechanisms.

In certain systems, pathologists are required to manually evaluate relative area of the sample that comprises the histologic features of interest. Due to the limited availability of tools, this often involved a great deal of subjective analysis, both as to what exactly constitutes tumor bed, viable tumor, tumor stroma, necrosis, etc., and exactly what percentage of the tumor bed was made up of viable tumor versus tumor stroma or necrosis. Standards and training can be introduced, but ultimately, the evaluation of pathologic response and use of pathologic response as a viable clinical endpoint is limited due to the high likelihood of variability across studies, across pathologists within individual studies, and even as performed by an individual pathologists on different samples or different days.

Persons of skill in the art will recognize that there are numerous benefits to be achieved by a tool using machine-learning models to automatically recognize the presence of tumor bed in digital pathology images of collected samples, and the automatically determine the relative area of histologic features of interest within the area of the tumor bed. As an example only, one benefit is the more efficient use of resources as pathologists can instead shift to a reviewing workflow to assess the accuracy of the machine-learning models and improve the machine-learning model through iterative training. A single pathologist can review and revise the annotations and calculations performed by the machine-learning models to ultimately perform analysis of more samples in less time. Additionally, the results of the machine-learning models may be significantly more reproducible such that when applied to the same samples or to similar samples over time, the variation between assessments is less than the variation that may be expected with manual evaluation. Machine-assisted analysis may help to avoid or diminish the effects of pathologist fatigue and identify features less detectable to the human eye. Furthermore, persons of skill in the art will recognize that there are numerous benefits to be achieved by a tool to automatically retrieve measured values associated with meaningful histologic features of interest and to further automatically calculate averages of the values as measured (e.g., the weighted and non-weighted average percentage of a tumor bed comprising viable cells). A tool employing the techniques described herein may be used to aid researchers in the discovery of new surrogate endpoints for overall survival rates, which, as explained herein are ultimately a difficult measure of success to use to the high likelihood of conflating factors and the oftentimes long timeframe. A tool for the automated assessment of pathologic response using digital pathology image processing techniques may assist in confirmation of suspected surrogate endpoints and may also assist in discovering new endpoints by providing a standardized and easily-reviewed data set, which may be ripe for further analysis by human researchers or other machine-learning systems.

Using an automated tool for the evaluation of pathologic response such as that described herein, the data collected regarding each sample in a study may be easily standardized, with the tool acting automatically enforcing the terms of the standardization built around the machine-learning models. This eliminates the risks associated with using different evaluators, such as different pathologists, in order to evaluate a large number of samples. Furthermore, the automated enforcement of standardization may aid with clinical validation by reducing the appearance of measurement bias or error. Additionally, the automated tool for the evaluation of pathologic response may provide data for analysis of whether certain types or categories of data are more useful in a clinical setting than others. For example, the tool may assist in determining whether weighted averages or non-weighted averages provide more evaluative feedback or whether the ratio of certain types of histologic features are indicative of certain patient outcomes. This may be used in a feedback loop to change the structure of a study or of the machine-learning models themselves.

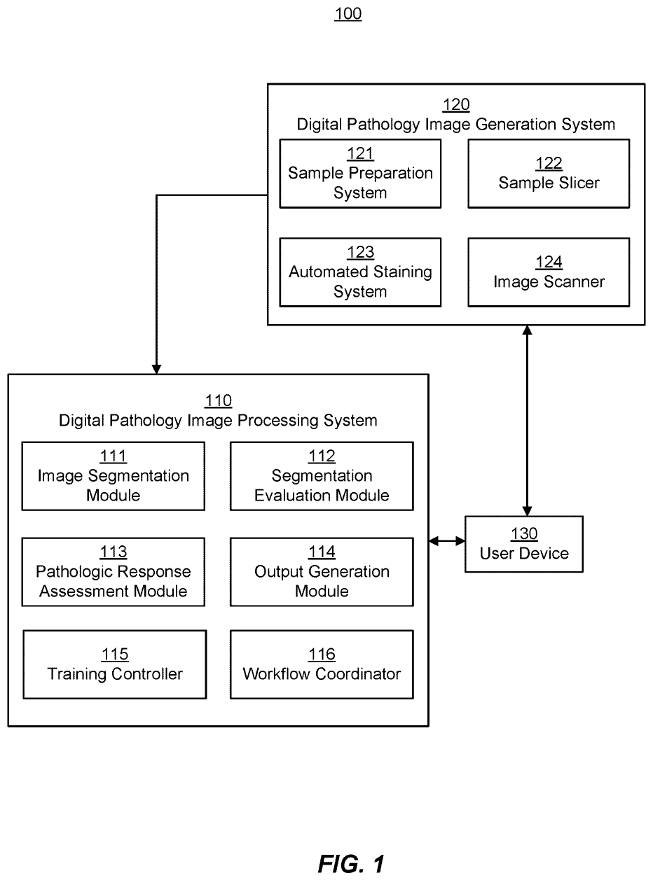

FIG. 1 illustrates a network 100 of interacting computer systems that may be used, as described herein according to some embodiments of the present disclosure.

A digital pathology image generation system 120 may generate one or more whole slide images or other related digital pathology images, corresponding to a particular sample. For example, an image generated by digital pathology image generation system 120 may include a stained section of a biopsy sample. As another example, an image generated by digital pathology image generation system 120 may include a slide image (e.g., a blood film) of a liquid sample. As another example, an image generated by digital pathology image generation system 120 may include fluorescence microscopy such as a slide image depicting fluorescence in situ hybridization (FISH) after a fluorescent probe has been bound to a target DNA or RNA sequence.

Some types of samples (e.g., biopsies, solid samples and/or samples including tissue) may be processed by a sample preparation system 121 to fix and/or embed the sample. Sample preparation system 121 may facilitate infiltrating the sample with a fixating agent (e.g., liquid fixing agent, such as a formaldehyde solution) and/or embedding substance (e.g., a histologic wax). For example, a sample fixation sub-system may fix a sample by exposing the sample to a fixating agent for at least a threshold amount of time (e.g., at least 3 hours, at least 6 hours, or at least 13 hours). A dehydration sub-system may dehydrate the sample (e.g., by exposing the fixed sample and/or a portion of the fixed sample to one or more ethanol solutions) and potentially clear the dehydrated sample using a clearing intermediate agent (e.g., that includes ethanol and a histologic wax). A sample embedding sub-system may infiltrate the sample (e.g., one or more times for corresponding predefined time periods) with a heated (e.g., and thus liquid) histologic wax. The histologic wax may include a paraffin wax and potentially one or more resins (e.g., styrene or polyethylene). The sample and wax may then be cooled, and the wax-infiltrated sample may then be blocked out.

A sample slicer 122 may receive the fixed and embedded sample and may produce a set of sections. Sample slicer 122 may expose the fixed and embedded sample to cool or cold temperatures. Sample slicer 122 may then cut the chilled sample (or a trimmed version thereof) to produce a set of sections. Each section may have a thickness that is (for example) less than 100 μm, less than 50 μm, less than 10 μm or less than 5 μm. Each section may have a thickness that is (for example) greater than 0.1 μm, greater than 1 μm, greater than 2 μm or greater than 4 μm. The cutting of the chilled sample may be performed in a warm water bath (e.g., at a temperature of at least 30° C., at least 350° C. or at least 40° C.).

An automated staining system 123 may facilitate staining one or more of the sample sections by exposing each section to one or more staining agents. Each section may be exposed to a predefined volume of staining agent for a predefined period of time. In some instances, a single section is concurrently or sequentially exposed to multiple staining agents.

Each of one or more stained sections may be presented to an image scanner 124, which may capture a digital image of the section. Image scanner 124 may include a microscope camera. The image scanner 124 may capture the digital image at multiple levels of magnification (e.g., using a 10× objective, 20× objective, 40× objective, etc.). Manipulation of the image may be used to capture a selected portion of the sample at the desired range of magnifications. Image scanner 124 may further capture annotations and/or morphometrics identified by a human operator. In some instances, a section is returned to automated staining system 123 after one or more images are captured, such that the section may be washed, exposed to one or more other stains, and imaged again. When multiple stains are used, the stains may be selected to have different color profiles, such that a first region of an image corresponding to a first section portion that absorbed a large amount of a first stain may be distinguished from a second region of the image (or a different image) corresponding to a second section portion that absorbed a large amount of a second stain.

It will be appreciated that one or more components of digital pathology image generation system 120 can, in some instances, operate in connection with human operators. For example, human operators may move the sample across various sub-systems (e.g., of sample preparation system 121 or of digital pathology image generation system 120) and/or initiate or terminate operation of one or more sub-systems, systems, or components of digital pathology image generation system 120. As another example, part or all of one or more components of digital pathology image generation system (e.g., one or more subsystems of the sample preparation system 121) may be partly or entirely replaced with actions of a human operator.

Further, it will be appreciated that, while various described and depicted functions and components of digital pathology image generation system 120 pertain to processing of a solid and/or biopsy sample, other embodiments may relate to a liquid sample (e.g., a blood sample). For example, digital pathology image generation system 120 may receive a liquid-sample (e.g., blood or urine) slide that includes a base slide, smeared liquid sample and cover. Image scanner 124 may then capture an image of the sample slide. Further embodiments of the digital pathology image generation system 120 may relate to capturing images of samples using advancing imaging techniques, such as FISH, described herein. For example, once a florescent probe has been introduced to a sample and allowed to bind to a target sequence appropriate imaging may be used to capture images of the sample for further analysis.

A given sample may be associated with one or more users (e.g., one or more physicians, laboratory technicians and/or medical providers) during processing and imaging. An associated user may include, by way of example and not of limitation, a person who ordered a test or biopsy that produced a sample being imaged, a person with permission to receive results of a test or biopsy, or a person who conducted analysis of the test or biopsy sample, among others. For example, a user may correspond to a physician, a pathologist, a clinician, or a subject. A user may use one or one user devices 130 to submit one or more requests (e.g., that identify a subject) that a sample be processed by digital pathology image generation system 120 and that a resulting image be processed by a digital pathology image processing system 110.

Digital pathology image generation system 120 may transmit an image produced by image scanner 124 back to user device 130. User device 130 then communicates with the digital pathology image processing system 110 to initiate automated processing of the image. In some instances, digital pathology image generation system 120 provides an image produced by image scanner 124 to the digital pathology image processing system 110 directly, e.g. at the direction of the user of a user device 130. Although not illustrated, other intermediary devices (e.g., data stores of a server connected to the digital pathology image generation system 120 or digital pathology image processing system 110) may also be used. Additionally, for the sake of simplicity only one digital pathology image processing system 110, image generating system 120, and user device 130 is illustrated in the network 100. This disclosure anticipates the use of one or more of each type of system and component thereof without necessarily deviating from the teachings of this disclosure.

The network 100 and associated systems shown in FIG. 1 may be used in a variety of contexts where scanning and evaluation of digital pathology images, such as whole slide images or image portions relevant to evaluation of neoadjuvant therapy according to the techniques described herein, are an essential component of the work. As an example, the network 100 may be associated with a clinical environment, where a user is evaluating the sample for possible diagnostic purposes and/or for study purposes. The user may review the image using the user device 130 prior to providing the image to the digital pathology image processing system 110. The user may provide additional information to the digital pathology image processing system 110 that may be used to guide or direct the analysis of the image by the digital pathology image processing system 110. For example, the user may provide a prospective diagnosis or preliminary assessment of features within the scan. The user may also provide additional context, such as the type of tissue being reviewed. As another example, the network 100 may be associated with a laboratory environment where tissues are being examined, for example, to determine the efficacy or potential side effects of a drug In this context, it may be commonplace for multiple types of tissues to be submitted for review to determine the effects on the whole body of said drug. This may present a particular challenge to human scan reviewers, who may need to determine the various contexts of the images, which may be highly dependent on the type of tissue being imaged. These contexts may optionally be provided to the digital pathology image processing system 110.

Digital pathology image processing system 110 may process digital pathology images, including images produced according to the techniques described herein, to classify the digital pathology images, generate annotations for the digital pathology images, generate predictions or assessments based on the digital pathology images, and produce related output. As an example, the digital pathology image processing system 110 may process images of tissue samples or tiles of the whole slide images of tissue samples generated by the digital pathology image processing system 110 to identify and process segments of the digital pathology images that correspond to tumor bed and/or that correspond to particular histologic features or evidence of the particular histologic features. As an example, the digital pathology image processing system 100 may identify histologic features in the digital pathology image that correspond to viable tumor cells, regions of viable tumor, necrotic tumor cells, regions of necrosis, tumor stroma cells, regions of tumor stroma, or other specified histologic features in the corresponding tissue sample. The digital pathology image processing system 100 may use computer vision algorithms to segment the digital pathology image based on the histologic features identified in the digital pathology image. Additionally or alternatively, the digital pathology image processing system 100 may use one or more machine-learning models to perform its evaluations. In addition to segmenting the digital pathology image, the digital pathology image processing system 100 may make assessments of the physical characteristics of the sample based on properties of the digital pathology image that are provided to the digital pathology image processing system 100 independently or as metadata with the digital pathology image. The digital pathology image processing system may further make assessments of the area and/or volume of the sample corresponding to segmented regions of the digital pathology image.

The digital pathology image processing system 110 may include an image segmentation module 111 to perform the image segmentation processes as described herein. The image segmentation module 111 may use or perform computer vision techniques to identify and segment the digital pathology image, or working copies of the digital pathology image, into one or more image segments according to desired analysis to be perform. As an example, the digital pathology image may include images of a tumor and other histologic features. The image segmentation module 111 may segment a first working copy of the digital pathology image into multiple regions based on whether the region of the image is determined or predicted to correspond to the tumor bed or not. The image segmentation module 111 may further segment a second working copy into multiple regions based on whether the portion of the image is determined or predicted to correspond to one or more histologic features of interest. As an example, and as described herein, with respect to evaluating the pathologic response of a sample after certain treatments, the histologic features of interest may include viable tumor cells, necrosis, and tumor stroma. As embodied herein, the image segmentation module 111 may also perform the second segmentation on the first working copy such that the portions of the image corresponding to the tumor bed are further sub-segmented into regions corresponding to the histologic features of interest.

The image segmentation module 111 may comprise or use one or more trained machine-learning models to perform the image segmentation. As an example, the image segmentation module 111 may use a first machine-learning model to segment the image based on the regions of the digital pathology image that are determined, by the machine-learning module, to correspond to the tumor bed in the sample. The image segmentation module 111 may further use a second machine-learning model to segment the image based on the regions of the digital pathology image that correspond to one of the one or more histologic features of interest. As an example, the second machine-learning model may be configured to identify regions of the digital pathology image that correspond to viable tumor cells, necrosis, or tumor stroma. The second machine-learning model may further label the identified regions accordingly such that the output produced by the second machine-learning model includes coordinates or other designations of the digital pathology image and labels for whether the coordinates are associated with viable tumor cells, necrosis, or tumor stroma. To perform the image segmentations, the first machine-learning model and the second machine-learning model may perform a variety of analyses on the image or a subdivision (e.g., tile) of the image, including, but not limited to, edge detection, image heuristic analysis, image classification and comparison, object detection and classification, semantic segmentation, and instance segmentation.

As embodied herein, the image segmentation module 111 may further define or select from image segmentation processes depending on the type of histologic feature being evaluated or the histologic features being detected. For example, the image segmentation module 111 may be configured with awareness of the type(s) of condition that the digital pathology image processing system 110 will be assessing and may customize the segmentation of the digital pathology image according to the relevant tissue abnormalities to improve detection. For example, the image segmentation module 111 may determine that, when the tissue abnormalities include searching for inflammation or necrosis in lung tissue,

As embodied herein, the image segmentation module 111 may further refine the image segmentation processes for the digital pathology image using one or more color channels or color combinations. As an example, digital pathology images received by digital pathology image processing system 110 may include large-format multi-color channel images having pixel color values for each pixel of the image specified for one of several color channels. Example color specifications or color spaces that may be used include the RGB, CMYK, HSL, HSV, or HSB color specifications. The image segmentation may be defined based on segmenting the color channels and/or generating a brightness map or greyscale equivalent of each tile. For example, image segmentation module 111 may use a red channel image, blue color channel image, green color channel image, and/or brightness channel image, or the equivalent for the color specification used. As explained herein, segmenting the digital pathology images based on segments of the image and/or color values of the segments may improve the accuracy and recognition rates of the networks used to evaluate the digital pathology image according to the techniques described herein and to produce assessments of the image. Additionally, the digital pathology image processing system 110. e.g., using the image segmentation module 111, may convert between color specifications and/or prepare working copies of the digital pathology using multiple color specifications. Color specification conversions may be selected based on a desired type of image augmentation (e.g., accentuating or boosting particular color channels, saturation levels, brightness levels, etc.). Color specification conversions may also be selected to improve compatibility between digital pathology image generation systems 120 and the digital pathology image processing system 110. For example, a particular image scanning component may provide output in the HSL color specification and the models used in the digital pathology image processing system 110, as described herein, may be trained using RGB images. Converting the digital pathology image to the compatible color specification may ensure the digital pathology image may still be analyzed. Additionally, the digital pathology image processing system may up-sample or down-sample images that are provided in particular color depth (e.g., 8-bit, 1-bit, etc.) to be usable by the digital pathology image processing system. Furthermore, the digital pathology image processing system 110 may cause images to be converted according to the type of image that has been captured (e.g., fluorescent images may include greater detail on color intensity or a wider range of colors).

A segmentation evaluation module 112 of the digital pathology image processing system 100 may analyze the segmented digital pathology image after processing by the image segmentation module 111. The segmentation evaluation module 112 may analyze the segmented images to determine, for example, the relative area of each identified segment to the area of other identified segments, the image, and/or the sample as a whole. As an example, the segmentation evaluation module 112 may calculate the area of the segmented image produced by the first machine-learning module that is determined to relate to the tumor bed. To do so, the segmentation evaluation module 112 may determine the number of pixels in the digital pathology image that correspond to the region of the segmented image. The segmentation evaluation module 112 may further determine the remaining number of pixels in the digital pathology image that correspond to the sample. The segmentation evaluation module 112 may then take the ratio of the two numbers of pixels to determine a percentage of pixels in the digital pathology image that corresponds to the tumor bed. Through similar process, the segmentation evaluation module 112 may determine the number of pixels in the segmented digital pathology image that correspond to, e.g., viable tumor cells, necrosis, and tumor stroma. The segmentation evaluation module 112 may compare the number of pixels corresponding to each type of histologic feature to the number of pixels in the digital pathology image overall. As discussed herein, another useful denominator may be the number of pixels corresponding to just the tumor bed (e.g., not including other histologic features that are not directly relevant to the evaluation of the tumor). The result of the analysis performed by the segmentation evaluation module 112 may be a series of ratios of the relative area of the relevant image segments. For example, the output may include a percentage of the digital pathology image (or the sample or even the tumor bed in the sample) corresponding to viable tumor cells, a percentage corresponding to necrosis, and a percentage corresponding to tumor stroma.

In addition or as an alternative to the using the number of pixels as an approximation of the relative areas of different image segments, the digital pathology image processing system 100, e.g., through the segmentation evaluation module 112, may calculate an actual area of the sample corresponding to the segments in the digital pathology image. As an example, the digital pathology image may be provided to the digital pathology image processing system with metadata including the dimensions of the sample corresponding to the digital pathology image. Additionally or alternatively, the digital pathology image may be provided with metadata corresponding to how the digital pathology image was created including the types of imaging equipment used (e.g., the name and/or model of a microscope or camera used) and the settings of the imaging equipment (e.g., pixel density, magnification settings). Using the metadata, the segmentation evaluation module 112 may further determine an approximate size of the sample corresponding to each pixel or to a specified area of the digital pathology image in order to provide image scale information. In combination, the image scale information may be used together with the identified segments to determine the size of the sample area and/or the sizes of various segments of the samples.

A pathologic response assessment module 113 may evaluate the output of the segmentation evaluation module 112 to determine a level or degree of a specified pathologic response based on the values determined by the segmentation evaluation module. The output of the pathologic response assessment module 113 may be the determination of whether the requirements for one or more specified conditions are satisfied. As an example, the pathologic response assessment module 113 may evaluate the relative percentages of the sample that correspond to each type of histologic feature. The pathologic response assessment module 113 may compare these relative percentages to one or more predetermined thresholds. The thresholds may be set by the user who requested evaluation of the digital pathology image, by a user who sets the standards for a clinical trial or study or may be set automatically by the digital pathology image generation system according to best practices relevant to the type of histologic feature being evaluated. As discussed herein, the percentages may be associated with a level of pathologic response. In the case of certain cancers, a percentage of the sample comprising viable tumor cells of less than 10% may be associated with MPR, while a percentage of the sample comprising viable tumor cells of less than 1% may be associated with pCR. In other cancers the percentages may vary, for example, a percentage of the comprising viable tumor cells less than 30% may be associated with MPR. In still other cases, the pathologic response assessment module 113 may compare the percentages of the sample corresponding to two or more types of histologic features to thresholds in making the assessment. In certain cases, other types of predetermined thresholds may be associated with other levels or classifications of pathologic response, such as, by way of example and not limitation, when a percentage of the sample characterized as regions of tumor necrosis meets and/or exceeds a specified threshold.

In particular embodiments, the digital pathology image processing system 110 may process multiple images together as corresponding to a single sample. As an example, a volumetric sample may be resected from a patient. The sample may be sliced into multiple sections and digital pathology images may be taken of each slice. Rather than perform a series of discrete analyses on each slice independently, the digital pathology image processing system 110 may perform a holistic analysis by combining the various area measures (e.g., the area of a digital pathology image corresponding to tumor bed or to one or more specific histologic features) into corresponding volume measures. Thus, the digital pathology image processing system 110 may prompt digital pathology image processing system 110 to process a series of images independently or jointly and may further store the various areal results for use in a final calculation by the pathologic response assessment module 113. The joint processing mode may be particularly useful where individual samples may create a misleading picture of overall MPR assessment. For example, a single image may include a high percentage of viable cells, but several other images may include a low percentage. Accordingly, the response assessment module 110 may adjust its overall assessment based on the volume of the sample calculated.

An output generation module 114 may produce various forms of output associated with the pathologic response assessment and the segmented digital pathology image. The output may indicate the level or degree of one or more types of pathologic response and the level or degree to which one or more requirements associated with the types of pathologic response were met. The output generation module 114 may generate output based on user request. As described herein, the output may include a variety of visualizations, interactive graphics, and reports based upon the type of request and the type of data that is available In many embodiments, the output will be provided to the user device 130 for display, but in certain embodiments the output may be accessed directly from the digital pathology image processing system 110. The output will be based on existence of and access to the appropriate data, so the output generation module 114 will be empowered to access metadata and anonymized patient information as needed. As with the other modules of the digital pathology image processing system 110, the output generation module 114 may be updated and improved in a modular fashion, so that new output features may be provided to users without requiring significant downtime.

A training controller 115 of the digital pathology image processing system 110 may control training of the one or more machine-learning models and/or functions used by the digital pathology image processing system 110. In some instances, some or all of the models and functions are trained together by training controller 115. In some instances, the training controller 115 may selectively train the models using by the digital pathology image processing system 110. For example, the digital pathology image processing system 110 may use a preconfigured model or pre-annotated samples to generate image segments corresponding to tumor beds and allow training to focus on training a second machine-learning model to segment the digital pathology image as corresponding to specific histologic features.

As embodied herein, the training controller 115 may select, retrieve, and/or access training data that includes a set of digital pathology images. The training data may further include a corresponding set of labels and/or annotations for particular histologic features in shown in the digital pathology images. During training operations, e.g., for the first machine-learning model or the second machine-learning model, the training controller 115 may cause the image segmentation module 111 to segment a subset of the digital pathology images in the training data. The output for each of the digital pathology images may be compared to the annotations and/or labels for the training data. Based on the comparison, one or more scoring functions may be used to evaluate the levels of precision and accuracy of the machine-learning model under testing. The training process will be repeated many times and may be performed with one or more subsets or cuts of the training data. For example, during each training cycle, a randomly-sampled selection of the digital pathology images from the training data may be provided as input to image segmentation module 111.

As an example, training controller 115 may use a scoring function that penalizes variability or differences between the provided annotations or labels and about the output generated by the image segmentation module 111. The scoring function may penalize differences between a distribution of annotations generated for each random sampling and a reference distribution. The reference distribution may include (for example) a delta distribution (e.g., a Dirac delta function) or a uniform or Gaussian distribution. Preprocessing of the reference distribution and/or the annotation location distribution may be performed, which may include (for example) shifting one or both of the two distributions to have a same center of mass or average. The scoring function may characterize the differences between the distributions using (for example) Kullback-Leibler (KL) divergence. If the distribution included multiple disparate peaks, the divergence with a delta distribution or uniform distribution may be more dramatic, which may result in a higher penalty. Other appropriate scoring functions may also be used. Scoring functions may be devised, for example, to incentivize the system to learn to identify specific structures and/or specific criteria indicative of the presence of structures as described herein. The results of the scoring function may be provided to the machine-learning model being trained, which applies or saves modifications to the model network to optimize the scores. After the model is modified, another training cycle begins with a new randomized sample of the input training data.

The training controller 115 determines when to cease training. For example, the training controller 115 may determine to train the machine-learning models or other algorithms used in the image segmentation module 11 for a set number of cycles. As another example, the training controller 115 may determine to train the image segmentation module 111 until the scoring function indicates that the models have passed a threshold value of success. As another example, the training controller 217 may periodically pause training and provide a test set of digital pathology images where the appropriate annotations are known. The training controller 115 may evaluate the output of the image segmentation module 111 against the known annotations to determine the accuracy of the image segmentation module 111. Once the accuracy reaches a set threshold, the training controller 115 may cease training.

A workflow coordinator 116 of the digital pathology image processing system 110 may control management integration of the digital pathology image processing system 110 into one or more digital image processing workflows, for example at the request of one or more users. The general techniques described herein may be integrated into a variety of tools and use cases. For example, as described, a user (e.g., pathology or clinician) may access a user device 130 that is in communication with the digital pathology image processing system 110 and provide a query image requesting analysis. The digital pathology image processing system 110, or the connection to the digital pathology image processing system may be provided as a standalone software tool or package that evaluates provided digital pathology images and provides output such as annotations and overall assessment evaluations. As a standalone tool or plug-in that may be purchased or licensed on a streamlined basis, the tool may be used to augment the capabilities of a research or clinical lab. Additionally, the tool may be integrated into the services made available to the customer of digital pathology image generation systems. For example, the tool may be provided as a unified workflow, where a user who conducts or requests an image to be created from a sample automatically receives a report including annotations generated from the segmented image, relative areas of histologic features of interest, and overall assessments based on the histologic features. This procedure may operate while the samples are being prepared, allowing for timely adjustment to sample preparation, e.g., to ensure that evaluations are consistent and useful. Therefore, in addition to improving digital pathology image analysis with respect to assessment of pathologic response, the techniques may be integrated into existing systems to provide additional features not previously considered or possible. The workflow coordinator 116 may manage the connections and interactions between the digital pathology image processing system 110 and the digital pathology image generation system 120, user device 130, and other servers or networks to which the digital pathology image processing system 110 may be coupled.

As an example, the digital pathology image processing system 120 may be used in digital or machine-assisted pathology reviews. The digital pathology image processing system 120 may be provided as a standalone tool having components for automated assessment of samples or may be in communication with other suites of tools that are specialized for review a particular type of samples. For example, the digital pathology image processing system 120 may access a library for assessing lung cancer tumor samples for a first workflow and access a library for assessing breast cancer tumor samples for a second workflow. In both cases, the digital pathology image processing system 120 may assist in automating review of samples, identifying areas of interest, and determining whether further review should be recommended.

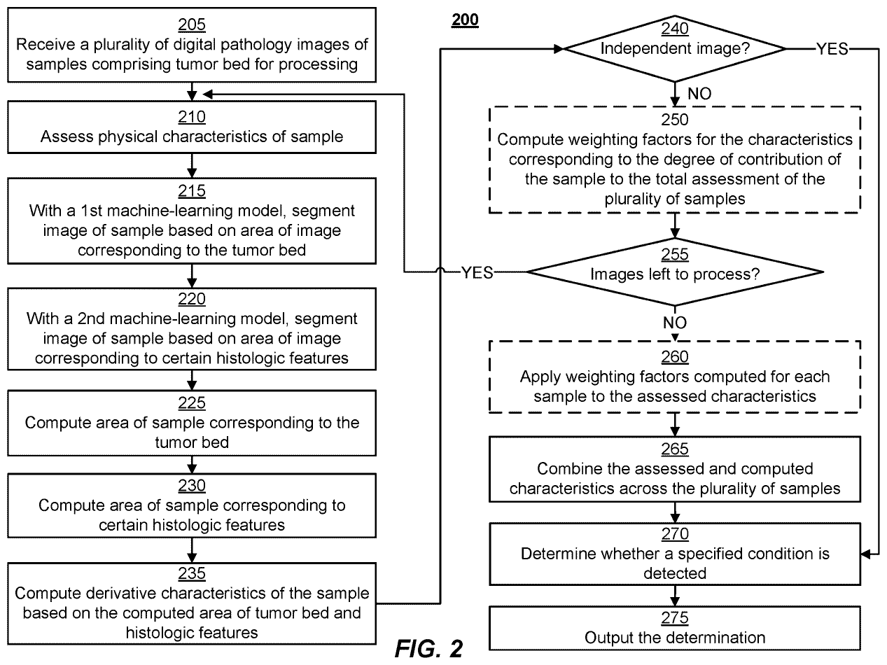

FIG. 2 illustrates an example process 200 for detecting one or more specified conditions, such as a particular type of pathologic response, in a sample based on assessment of a digital pathology image. The process may begin at step 205, where the digital pathology image processing system 110 receives one or more digital pathology images of samples for processing. Each digital pathology image may include a tumor bed in at least a portion of the digital pathology image. The digital pathology images may include a plurality of independent images (e.g., unrelated through a single sample) or may be grouped as from a single resection or evaluation event (e.g., the entire plurality of samples is from the same master sample).

At step 210, the physical characteristics of a sample as captured in an individual digital pathology image are assessed. The assessment may be performed directly by the digital pathology image processing system. For example, the physical characteristics of the sample may include the two-dimensional or three-dimensional dimensions of the sample. To assess the characteristics of the sample as shown in the digital pathology image, the digital pathology image processing system 110 may reference metadata provided in or with the digital pathology. As an example, the metadata may include information related to the generation of the digital pathology image 110, such as the pixel density of the image or the image sensor, the magnification levels used in generating the digital pathology image, etc. In particular embodiments, the physical characteristics may be initially provided by an operator or by the digital pathology image generation system 120.

At step 215, the digital pathology image processing system 110 may segment the digital pathology image based on the area of the sample shown in the digital pathology image corresponding to the tumor bed. The segmentation may be performed, for example, by the image segmentation module 111. The image segmentation module 111 may use, for example, a first machine-learning model trained to characterize or predict regions of a digital pathology image as corresponding to the tumor bed of a particular sample. The first machine-learning model may be trained to recognize variations in color, structures within the image, and other signs of the tumor bed and classify regions of the image accordingly. In particular embodiments, the image segmentation module 111 may produce a new instance of the digital pathology image including annotations corresponding to the segmented regions (e.g., to prevent the original digital pathology image from being permanently altered or destroyed).

At step 220, the digital pathology image processing system 110 may segment the digital pathology image based on the area of the sample shown in the digital pathology image corresponding to one or more predetermined types of histologic features (e.g., viable tumor cells, necrosis, and stroma). The segmentation may be performed, for example, by the image segmentation module 111. The image segmentation module 111 may use, for example, a second machine-learning model trained to characterize or predict regions of a digital pathology image as corresponding to each of the predetermined histologic features in a tumor bed. The second machine-learning model may be trained to characterize regions of the digital pathology image corresponding to predetermined histologic features associated with a specific type of assessment. The second machine-learning model may be trained to recognize variations in color, structures within the image, and other signals within the image as indicative of certain types of histologic features. As an example, when assessing pathologic response by cancerous lung tissue to certain treatments, the second machine-learning model may be configured to identify regions of the digital pathology image corresponding to viable tumor cells, tumor stroma, and necrosis. In particular embodiments, the image segmentation module 111 may produce a new instance of the digital pathology image including annotations corresponding to the segmented regions (e.g., to prevent the original digital pathology image from being permanently altered or destroyed). In particular embodiments, steps 215 and 220 may be collapsed into a single step by utilizing a single machine-learning model trained to characterize regions of the digital pathology image corresponding to tumor bed as well as the predetermined histologic features.

At step 225, based on the segmented digital pathology image, the digital pathology image processing system 110 may compute the area of a sample shown in the digital pathology image corresponding to the tumor bed. The evaluation may be performed by a segmentation evaluation module 112. As described herein, the segmentation evaluation module 112 may determine the number of pixels or size of the region of the digital pathology image that may been segmented as corresponding to the tumor bed. The segmentation evaluation module 112 may determine an associated area of the sample based on the size of the digital pathology image. As an example, the segmentation evaluation module 112 may use the physical characteristics of the sample and/or use metadata associated with the digital pathology image to compute the area of the sample.

At step 230, based on the segmented digital pathology image, the digital pathology image processing system 110 may compute the area of a sample shown in the digital pathology image corresponding to the predetermined histologic features. The evaluation may also be performed by the segmentation evaluation module 112. As described herein, the segmentation evaluation module 112 may determine the number of pixels or size of the region of the digital pathology image that may been segmented as corresponding to each of the predetermined histologic features. Based on the size of the regions of the digital pathology image, the physical area of each of the regions may be determined.

At step 235, the digital pathology image processing system 110 may compute characteristics of interest of an individual sample are assessed. The characteristics of interest may be considered derivative characteristics under evaluation, as they may be computed from the directly measured values, such as the areas of the segmented cell types- and/or regions-of-interest of the sample. In particular embodiments, the characteristics of interest may be based on the type of histologic feature being evaluated, which information may be provided to the digital pathology image processing system 110. As an example, the characteristics of interest may include the relative sizes of the areas of the digital pathology image, and thus the sample, corresponding to each of the predetermined histologic features. In the example of evaluation of response of lung tissue samples to certain treatments, the characteristics may include relative area, within the digital pathology image, of each of a viable tumor cells, tumor stroma, and necrosis. In addition to the relative sizes, the characteristics may further include the relative percentages of the sample area and/or of the tumor bed, that are characterized as each of the predetermined histologic features. While the areas and other characteristics may be computed directly based on the association of the size of regions in the digital pathology image to physical dimensions, characteristics may also be derived based on assumptions regarding the relationship between certain types of histologic features. For example, under the assumption that the tumor bed captured in a digital pathology image may comprise one of three types of histologic features (e.g., viable tumor cells, necrosis, and tumor stroma), once the areas of two of the types of histologic feature are known and the area of the tumor bed is known, the area of the third type may be derived. Similar principles may be used to compute the relative percentages of the tumor bed that comprise each of the predetermined histologic features.

At step 240, the digital pathology image processing system 110 may determine whether the digital pathology image under analysis is an independent image or is part of a collection of digital pathology images that are related to the same sample or group of samples (e.g., taken from a master sample). The digital pathology image processing system 110 may be configured to process the samples based on whether they are to be treated independently or not. As an example, the workflow coordinator 116 may determine or instruct the digital pathology image processing system on how to proceed. If the determination at 240 is that the image is an independent image, the process advances to step 270 where an assessment of the digital pathology image is made. If the determination at step 240 is that the image is not an independent image, the process advances to step 250.

At step 250, the digital pathology image processing system 110 may compute weighting factors for the characteristics that have been assessed. The weighting factors may be selected and determined to correspond to the degree of contribution of the individual sample to the total assessment of the plurality of samples (e.g., the master sample). The weighting factors may be based on the area of the individual sample relative to the total sample area of the plurality of samples. The purpose of the weighting factors may be to ensure that each individual digital pathology image contributes to the overall assessment of the sample to the degree that it is representative of the sample. For example, a digital pathology image with a smaller overall area of the sample or tumor bed, but a higher percentage of a certain type of histologic feature may be given lower weighting factors so as to not improperly skew the results towards that type of histologic feature. Other methods of determining a weighting factor may also be used.

At step 255, the digital pathology image processing system, e.g., through the workflow coordinator 116 may determine whether there are additional digital pathology images of the plurality of digital pathology images yet to be processed for the group of digital pathology images related to the sample. If so, the process returns to step 210 to process additional image. If there are no more images to be processed for a given sample, the process proceeds to step 260.

At step 260, if weighing factors were computed, the digital pathology image processing system 110 applies the weighting factors computed for each digital pathology image to the assessed characteristics of each digital pathology image. For example, the weighting factors may comprise scaling factors that may be applied to one or more of the characteristics of interest before they are combined. At step 265, the digital pathology image processing system 110 combines the assessed and computed characteristics across the plurality of digital pathology images. As example, the assessed and computed characteristics may be combined in a weighted combination (e.g., based on the determined weighing factors). Additionally or alternatively, the assessed and computed characteristics may be combined by averaging the values of each characteristic across the plurality of samples.

At step 270, the digital pathology image processing system 110 generates an assessment regarding a specified condition. In particular, a pathologic response assessment module 113 determines whether a specified condition is detected in the image, or in the case of a collection of digital pathology images being provided, is detected in the plurality of images. As an example, the determination may be based on whether one or more of the combined characteristics satisfy a certain threshold. The threshold may be set based on, for example, the amount or quality of the images, the physical characteristics of the samples depicted in the images, the type of tissue being assessed, or the type of condition being assessed. Various threshold may be used for the various characteristics. For example, an assessment may be rendered based on the relative percentage of the tumor bed comprises viable cells. If the percentage satisfies a first threshold (which may be based on the type of sample depicted in the digital pathology image), then a first type of assessment may be determined (e.g., MPR detected) if the percentage satisfies a second threshold, then a second type of assessment may be determined (e.g., pCR detected). Combinations of the characteristics may also be assessed together. As an example, if the percentage of the tumor bed includes a first type of histologic feature and the area of the sample bed satisfy certain thresholds or other requirements, a specified type of assessment may be determined.

At step 275, the digital pathology image processing system 110 prepares and provides an output corresponding to the determination of the assessment. The output generation module 114 may prepare a variety of types of outputs corresponding to the assessment. For example, the output may include a plain language statement of the assessment (e.g., “MPR Determined”) and/or may include a listing of relevant characteristics that led to the assessment (e.g., “MPR Determined; Viable Tumor 8%”). The plain language statement may be incorporated into a report or user interface providing additional details regarding the digital pathology image and/or sample. Additionally or alternatively, visualizations may be generated for the output, such as a visualization illustrating the various segmented portions of the digital pathology image that were used in making the assessment. Taken together, the outputs may be used to provide the assessment determined by the digital pathology image processing system 110 as well as provide insight into how the assessments were made, permitting operators to validate the results and provide feedback to improve the digital pathology image processing system 110 (e.g., where the feedback is used by the training controller 115 to update the first or second machine-learning models).

Particular embodiments may repeat one or more steps of the process of FIG. 2, where appropriate. Although this disclosure describes and illustrates particular steps of the process of FIG. 2 as occurring in a particular order, this disclosure contemplates any suitable steps of the process of FIG. 2 occurring in any suitable order. Moreover, although this disclosure describes and illustrates an example process for automated assessment of a digital pathology image for pathologic response including the particular steps of the process of FIG. 2, this disclosure contemplates any suitable process for the same including any suitable steps, which may include all, some, or none of the steps of the process of FIG. 2, where appropriate. Furthermore, although this disclosure describes and illustrates particular components, devices, or systems carrying out particular steps of the process of FIG. 2, this disclosure contemplates any suitable combination of any suitable components, devices, or systems carrying out any suitable steps of the process of FIG. 2.

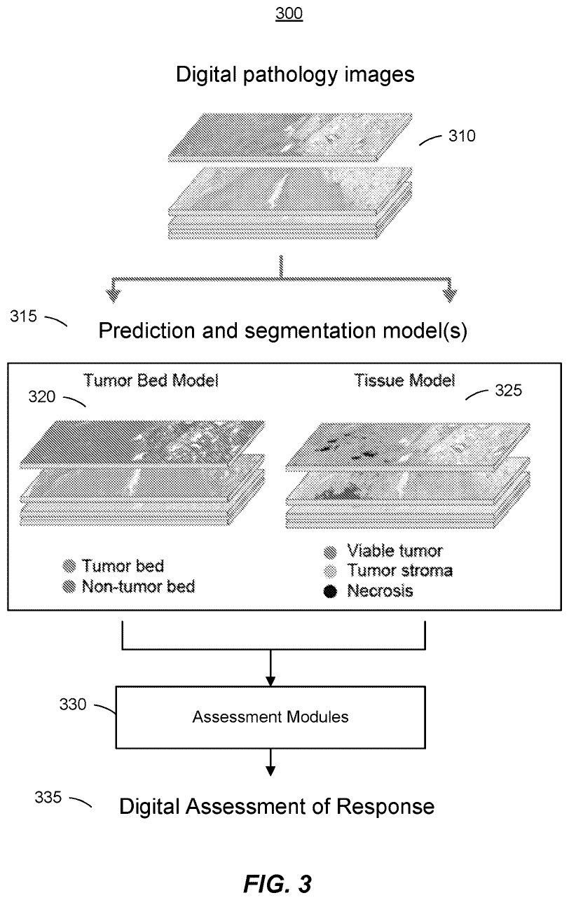

FIG. 3 illustrates a schematic overview 300 of the use of machine-learning models for assessing tissue response to certain types of therapies. As described the input to the model for assessing the response may include one or more digital pathology images 310. In particular embodiments, the digital pathology images may include images of H&E-stained samples. The digital pathology images 310 are provided to one or more prediction and segmentation models 315. In particular embodiments, the model 315 trained to evaluate digital pathology images for digital MPR assessment may comprise two co-trained models. A first model 320 may be trained to determine the portions of an image that comprise the tumor bed and the portion of the image that comprise other areas (e.g., reactive inflammatory tissue or image artifacts). In particular embodiments, the tumor bed may comprise the stroma, viable tumor cells, necrosis, and other types of histologic features that are associated with a tumor bed. The tumor bed model 320 may be trained to receive a digital pathology image 310, such as a digitized H&E-stained image, and annotate or otherwise indicate which regions of the image include the tumor bed and which regions of the image do not. The tumor bed model 320 for identifying the tumor bed may be trained, for example, to recognize predetermined histologic features and other visual features of the sample in the digital pathology image when evaluating which regions of the digital pathology image correspond to tumor bed and which regions do not.

A second model 325 may be trained to determine cell types- and/or regions-of-interest based on the condition being evaluated. As described herein, in the case of digital MPR assessment for lung cancers, the cell types- and/or regions-of-interest may include viable tumor cells, tumor stroma, and necrosis. The tissue model 325 may be trained to receive the same digital pathology image 310 and annotate or otherwise indicate which regions of the image include viable tumor cells, tumor stroma, and necrosis. The tissue model 325 may further determine the area of each type of cell within the tumor bed (e.g., the area of the tumor bed that comprises viable tumor cells, the area of the tumor bed that comprises tumor stroma, and the area of the tumor bed that comprises necrosis). As discussed, these cells contribute to the tumor bed, so the total area of each of the determined regions is expected to equal the area of the tumor bed. The relative area of each region is calculated as the area of the region divided by the total area of the tumor bed. The tissue model 325 for identifying different cell types- and/or regions-of-interest may also be trained to recognize predetermined histologic features and other visual features that are indicative of whether a given region of the digital pathology image correspond to, for example, viable tumor cells, tumor stroma, and necrosis.

In combination, the first model 320 and second model 325 analyze and provide feedback on features within the digital pathology image 310 that are relevant to pathologic response assessment. The models 315 give a determination relevant to different types of assessment categories such as major pathologic response (MPR) and pathologic complete response (pCR). The predictions and segmentations generated by the prediction and segmentation models 315 may be provided to the assessment modules 330 which interpret the output of the tumor bed model 320 and the tissue model 325 and generate actionable assessment of the response 335 therefrom.

As described herein, MPR assessment performed manually is an arduous and highly subjective task. Therefore, generating true high quality training data from existing samples may be difficult. Training data may be obtained from existing clinical studies or assessments where initial pathologist work was reviewed and confirmed or corrected by a team of reviewing pathologists who agreed on the eventual assessment. As the machine-learning models are trained based on the annotations and assessments within the training data, the accuracy of the training data will directly influence the accuracy of the model. As such, annotations relating to individual slides (e.g., where each sample has multiple slides) may be separated and treated independently In addition, the inclusion of pre-analytical variables, such as scanning format, magnification, tissue quality, and stain color stability may increase the availability of trustworthy ground truth data. As additional data is made available, the training regime may transition to training across slides as a whole, enabling the model to derive insight relating to the entire assessed volume of a tumor bed and not just the area depicted in an individual slide.

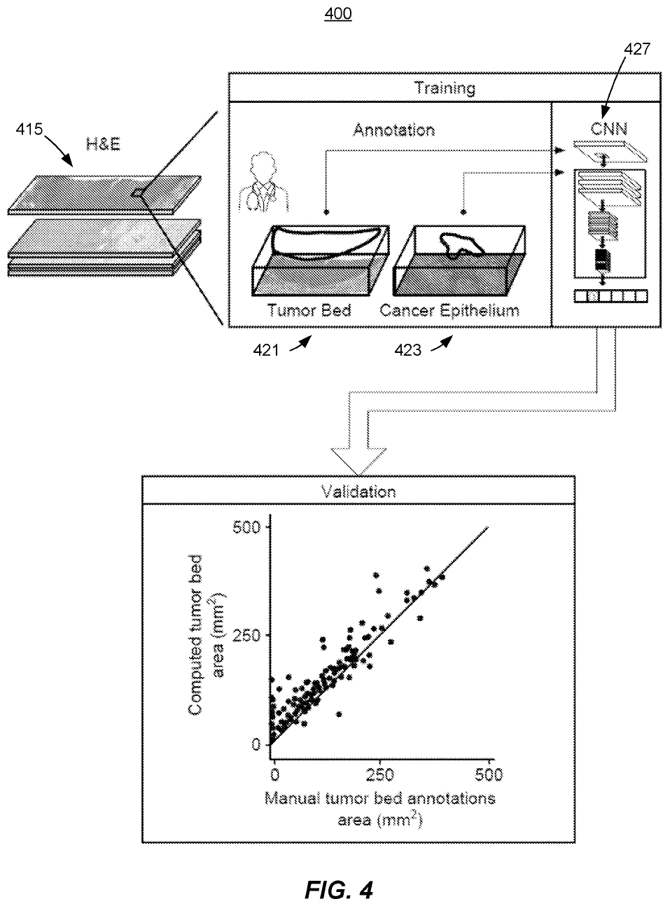

FIG. 4 illustrates an example process 400 for training models to be incorporated into a tool for automated assessment of major pathologic response. During pre-processing, digital pathology images 415 may be collected and/or created. During a training phase, annotated versions of the digital pathology images demarcating specific regions of interest (e.g., tumor bed 421 or viable tumor 423) may be provided (e.g., by a pathologist or a consensus group of pathologists) with the digital pathology images 415 to a convolutional neural network (CNN) 427, in order to train CNN 427 to recognize cell types-of-interest, cell morphology, and other regions-of-interest and appropriately annotate the digital pathology images accordingly. During a validation phase, trained CNN 427 may be applied to digital pathology images 415 again in order to validate the trained model and ensure that its outputs correspond to the annotations used to train the model.

The training data 415 that is provided may include a plurality of annotated digital pathology images (e.g., H&E-stained tissue images). The annotations may include an outline of the regions of the image that correspond to histologic features of a particular cell type and structure. As an example, annotations provided with the digital pathology images may include an indication of which regions of the image correspond to tumor bed, viable tumor, tumor stroma, and necrotic tumor. The annotations may include metadata relating to how the image was captured that may be used to derive the real size of the area (e.g., the area of the physical sample).

Additionally or alternatively, the digital pathology image data 415 (including annotations for training purposes) may be augmented with other types of imaging data, such as digital radiographic images. These other types of image data may be used as they may provide indications that are not typically present in digital pathology images and may be useful for a model in automatically assessing certain histologic features. Additionally or alternatively, beyond just visual data alone, features derived from the digital pathology images, the other types of images, and/or biomarker data from tissue or blood may be used to augment the image data.

Training models to accurately (e.g., with high precision and recall) identify the histologic features of interest may be based on the provision of as broad an understanding of the tools used by pathologists for similar assessments. Training may include manually annotating results to provide further feedback for the models. This additional step of refinement may enable the training regime of the model to learn to a more nuanced degree. As an example, the model may initially be trained via supervised or unsupervised methods to learn to identify particular histologic features based on the training set of annotated images. After a threshold accuracy or number of iterations has been reached, output form the model may be provided to a reviewer, such as one or more pathologists. The reviewer may annotate the output to indicate areas of potential improvements. For example, the reviewer may indicate through the annotations that area in the digital pathology image is not properly marked as tumor bed, or that a cluster of cells should not be marked as viable tumor. When making annotations, the reviewer may supply justification for the error. The annotation and justification may serve as training data for a revised training set of data. The revised training set of data may assist the training of the model to learn best practices or learn to identify rare scenarios.

As described above, the annotations, either provided initially or during a mid-training or subsequent review, may include specifying an area that is improperly labeled (e.g., as tumor bed or not tumor bed). Annotations may also indicate specific histologic features. For example, the tumor bed may be identified through the identification of histologic changes in the tumor sample associated with the neoadjuvant therapy. The border of the tumor bed may be identified by the geographic transition from tumor-associated stroma with treatment-associated changes to non-tumor-associated connective tissue and organ parenchyma. In some cases, the architecture of the lung is preserved with interstitial thickening by fibrosis and inflammation indicative of the tumor bed. The extent of the tumor bed may be estimated by identifying common tumor and treatment associated changes, such as organizing pneumonia, marked type II pneumocyte hyperplasia or reactive atypia, and types of inflammatory infiltrates including chronic or acute inflammation, histiocytes, giant cell reaction, and granulomas. In particular embodiments, the tumor bed may be distinguished from the reactive changes in the surrounding lung parenchyma by identifying preserved underlying alveolar architecture or non-tumor pathologies, while in the tumor bed the lung architecture is destroyed.

Necrosis may include completely necrotic tissue or may be filled with neutrophils or other inflammatory cells. Other features may be further detected as indicative of necrotic tissue in the digital pathology image such as cholesterol clefts, features indicative of focal necrosis, organized death of cells or unorganized death of cells (clumping of dead cells indicates how they died), treatment associated necrosis, granulomas; coagulation necrosis, or foam cell infiltration.

Tumor stroma may comprise, for example, dense hyalinized fibrosis, fibroelastotic scars, myofibroblastic cells or capillary-sized blood vessels. The following features may be detected as indicative of stromal tissue in the digital pathology image: degrees of inflammation associated with fibrosis, grade of inflammation; treatment associated fibrosis, inflammatory deposits such as lymphoid aggregates; inflammatory infiltrates; or acinar glands and the adjacent stroma showing chronic inflammation and loose myxoid connective tissue.

When evaluating viable tumor cells, best practices that may be trained into the model include that only well-preserved tumor cells should be regarded as tumor cells for the purpose of assessing MPR. Certain specific types of cancers may have additional rules or best practices, for example, for colloid adenocarcinomas, mucin pools should be included in the percentage of viable tumor. However areas of extracellular mucin without any viable tumor cells within the mucin may be regarded as stromal tissue.

In addition to these best practices, annotations may provide to the training data set 415 information regarding specific histologic features that may be detected within the digital pathology image that may further reinforce the assessment of MPR or pCR. As described herein, the percentage of viable tumor cells is one factor in making an MPR assessment. Other factors may include the presence of specific histologic features and the heterogeneity of the histologic features. The histologic features may also be prognostically significant between types of cancers, even when the same percentage of viable tumor or other histologic features are present. Histologic features of interest beyond the identification of viable tumor, tumor stroma, and necrotic tumor may include fibroelastotic scars associated with lung cancers (particularly adenocarcinomas), vascular changes including inflammation of blood vessel walls or vasculitis, medial fibrotic thickening (which sometimes obliterates vascular lumens), recanalization, cytologic atypia (of the tumor cells), and relative dimensions of such histologic features.

Metadata regarding the treatment of the patient may be provided during training or refinement to further inform the assessment. For example, pharmacodynamic effects of targeted therapies and other kinds of therapies on the tumor microenvironment have been documented and may be provided for training to the model. During evaluation, metadata provided to the model with a digital pathology image may indicate the current or previous treatment regimens of the patient.

All of the above-mentioned histologic features may be embedded in the annotations provided to the training data on either initial training or refinement. The training controller 116 may be further trained to analyze the quantity and quality of data being input into the model(s) to assess best practices regarding data collection and presentation for accurate results for pathologic response assessment. As an example, the training controller 116 and control, and make recommendations or enforce rules regarding, for example, the number of slides presented to the model, the size of slides presented, the size of certain areas and/or regions for analysis, the total area and/or volume of samples when evaluating across slides.

In addition to training to identify particular histologic features or type, the machine-learning model may also be trained to validate existing analysis and to improve on analytical frameworks. For example, the machine-learning model may be used to test the parameters of a study such as the required number of samples, minimum area of tissue bed in a sample for it to be usable, the total area and/or volume of samples that must be available to make an MPR assessment, the magnification level used to generate digital pathology images, etc. Additionally or alternatively, the highly reproducible nature of the machine-learning-based assessment allows for the exploration of the data to determine additional insights. For example, the biomarker thresholds for different types of cancers or treatment regimens may be quickly and easily evaluated when assessing MPR or pCR or other pathologic response. One could take data from clinical trials and explore a new cut-off based on digital reads for a specific disease and treatment setting as well as histology. This cut-off could be refined based on the addition of other biomarker data to develop a better surrogate for DFS, OS, RFS, EFS, or a particular type of pathologic response. The import of combinations or relative levels of different histologic features (e.g., the ratio of viable tumor to tumor stroma, or necrotic tumor to stroma) may be assessed by eliminating the bias and potential sampling error of subjective analysis by pathologists. In particular embodiments, the machine-learning model may also be able to detect, identify, evaluate, and/or measure certain biomarkers that may be useful in the evaluation of patient outcomes when combined with a particular type of pathologic response, such as other predictive/prognostic biomarkers from tissue or blood (e.g. determined by immunohistochemistry, mutation analysis, gene expression analysis), or histopathologic features specific to a disease or indicative of a treatment effect). For example, in a sample from a patient with ALK rearrangement prior to treatment (e.g., ALK IHC+) who is enrolled into a trial receiving neoadjuvant Alectinib, a lack of visible viable tumor cells in the digital pathology image may correspond to detection of very few ALK+viable tumor cells scattered throughout the tumor bed upon performing an ALK IHC test.

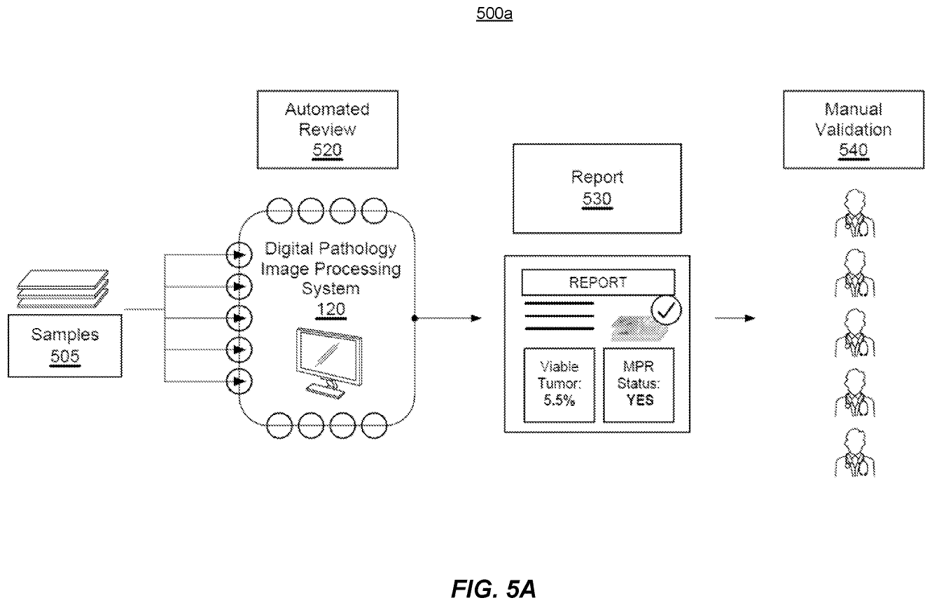

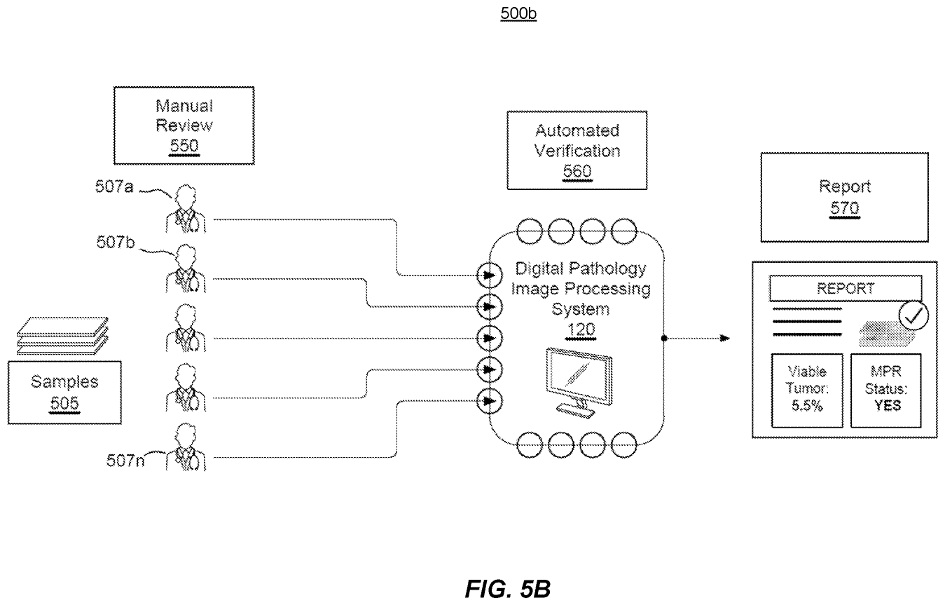

Examples will now be discussed illustrating potential workflows that may be provided using a digital MPR assessment model FIG. 5A illustrates a first workflow 500a in which the digital pathology image processing system 120 processes samples 505, using the techniques described herein. The samples are provided to and processed by the digital pathology image processing system 120 during an automated review stage 520. The output from the digital pathology image processing system 120 is provided during a reporting stage 530. The report may include information such as the overall assessment, the information derived from the samples that contributes to the assessment (e.g., the percentage of area of the sample(s) including viable tumor cells), annotated images versions of the digital pathology images of the samples, and other similar information.

Once the report is prepared, the report is provided to one or more pathologist reviewers for a manual validation stage 540. The pathologist reviewers may use the values in the report accordingly (e.g., as used in a clinical study, for evaluation of a patient). The pathologist reviewers may further ensure that the values included in the report appear correct. In the event that the values appear incorrect or the pathology disagrees with an assessment, the pathologist may provide corrections which may be used by the training controller 116 to update the appropriate models. The corrections may be provided, for example, as additional annotations on top of the images or re-evaluation of the text. In particular embodiments, the digital pathology image processing system 120 may associate a degree of confidence with each assessment to flag certain values for review by the pathologist. For example, if the determined percentages have an associated confidence level below a threshold confidence level, the digital pathology image processing system 120 may flag the values for manual review.

FIG. 5B illustrates a second workflow 500b in which the digital pathology image processing system 120 receives evaluations from one or more pathologists and performs a level of secondary review. The samples 505 are collected and/or provided to one or more pathologists 507a, 507b . . . 507n. During a manual review stage 550, the pathologists perform their own assessments of the one or more samples 505. The evaluations are then provided to the digital pathology image processing system 120 for an automated verification stage 560. The evaluations may include both the overall assessment of a sample (and/or the individual digital pathology images associated with the sample), the digital pathology images, and annotations generated by the pathologists 507a, 507b, and 507n for the digital pathology images. The annotations may indicate areas or regions of the digital pathology image that were relevant to the assessment provided by the pathologists 507a, 507b, and 507n.

In the second workflow 500b, the digital pathology image processing system 120 may ensure that all pathologists are adhering to a set of standards for evaluations. The standards may be selected based on the samples and the context of the evaluations of the samples, such as the type of tissue, the type of sample, or the type of assessment being performed. For example, the digital pathology image processing system 120 may generate its own assessments (including its own annotations) and compare the assessments to those provided by the reviewers. The digital pathology image processing system 120 may prepare a report during a reporting stage 570 that identifies and characterizes discrepancies between what the pathologists assessed for each sample and what the digital pathology image processing system 120 identified as the appropriate assessment. The report may include for example, a side-by-side comparison of the assessments, the derived characteristics, or the annotations prepared for a sample under evaluation by a pathologist and by the digital pathology image processing system 120.

As discussed herein, in addition to storing the results of individual samples and blocks, the data stored by the digital pathology image processing system 120 may be analyzed over time (and across clinical studies) to identify ongoing trends that may prove useful for providing clinical validation for the techniques discussed herein or to simply ensure that data is consistent. For example, by recording and collecting pathologist identification information for each sample assessment they make, trends may be identified in pathologist performance in assessing the true percentages or other values of interest. Additionally, the collected data may be provided in a relatively standardized format to the training controller 116 or other machine-learning systems as a well-conditioned dataset to facilitate automated learning. The automated learning may be used to evaluate the usefulness of proposed surrogate endpoints as well as to propose the study of additional surrogate endpoints. Similarly, cross-referencing may be performed in which multiple pathologists assess the sample set of samples as a way of directly comparing the assessment tendencies of the pathologists as compared to the results from the digital pathology image processing system 120. Because results are likely to be provide stronger evidence when the assessments are consistent, the digital pathology image processing system 120 provides the ability to track trends is assessments over time and to introduce repeatability to the analysis which was heretofore impracticable.

In addition, tracking information such as block identifying information and patient identifying information be used to track and compare clinical population results across a clinical study and potentially over time. For example, if a single patient submits multiple samples over time, tracking of dates and patient identifying information may be used to study effects of time and study the progress of a mass or tissue in an individual.

Although the description given herein results to certain specific types of assessments that have been determined to be useful for certain types of cancers, the digital pathology image processing system 120 may perform assessments with respect to a wide variety of surrogate endpoints and for evaluating a wide variety of conditions, including, but not limited to evaluating pathologic response of varying degrees in many varieties of cancers affecting many types of organs. Indeed, the digital pathology image processing system 120 may be able to assess multiple conditions, with customized indicators that are recorded, calculated, and stored by the digital pathology image processing system 120. For example, the digital pathology image processing system 120 may include a suite of data types to be analyzed, recorded and stored, including but not limited to area of the sample, volume of the sample (which may be extrapolated from measurements in multiple dimensions), mass of the sample, density of the sample, percentage of the sample (across one or more dimensions) comprising a type of cell or other biological entity or exhibiting a specified condition, oxygenation of the sample, and many others. Additionally, the digital pathology image processing system 120 may evaluate how the data types correlate with radiological assessments (e.g., based on CT scans) and biomarkers from other tests and assessments. The digital pathology image processing system 120 may perform different types of response assessments and share the assessments among pathologists and diagnosticians. In using the digital pathology image processing system 120 to establish a clinical study, the architect of the study may customize the digital pathology image processing system 120 as needed, using a library of values and calculations or recording her own for the study, Therefore, the digital pathology image processing system 120 may be used as a single, unifying interface for wide variety of clinical researchers, decreasing use pick-up time with reducing operator errors due to unfamiliar tools and interfaces and increasing the repeatability of results so that assessments are consistent across samples and performed in a greatly reduced time as compared with human evaluators.

As another example, particular embodiments of the digital pathology image processing system 120 may evaluate entered data to determine whether values for a given sample are unreasonable. Where values are determined to be likely to be unreasonable, (e.g., based on predetermined or automatically-learned thresholds or ranges), the digital pathology image processing system 120 may prompt an operator to correct any unexpected behaviors or, alternately, to confirm previous values. Furthermore, the digital pathology image processing system 120 may prevent, where possible, the entry of unreasonable values by automating determination of derivative values. Additionally or alternatively, the digital pathology image processing system 120 may evaluate for certain benchmark values based on known or expected correlations between values. For example, in a given clinical study it may be determined that samples of a given size are highly unlikely to have a certain condition present (e.g., a percentage of a tumor bed comprising necrosis greater than 80%). The digital pathology image processing system 120 may be configured for said study with this information and may detect when the unlikely condition is present. The digital pathology image processing system 120 may indicate the detection and calculation of this data as a potential error, prompting the operator to confirm the value, correct an error, or cause the digital pathology image processing system 120 to attempt to correct the error through further analysis. Additionally, or alternatively, the digital pathology image processing system 120 may compare new samples for a particular block (e.g., a new sample associated with a particular tumor), a new block for a given subject (e.g., a new tumor under study for a particular patient), or new block data for a given study (e.g., a new tumor under study associated with a clinical study) to previously determined values and determine whether a particular value is out of an expected range of values based on the historical data. The digital pathology image processing system 120 may prompt for further analysis by an operator or may flag the new data as being of potentially high interest to a clinical researcher. By analyzing entered data and prompting for further analysis, the digital pathology image processing system 120 may ensure consistency across individual operators and even multiple operators over time. Additionally, the digital pathology image processing system 120 may support blinded review of a pathologist and automated evaluations.

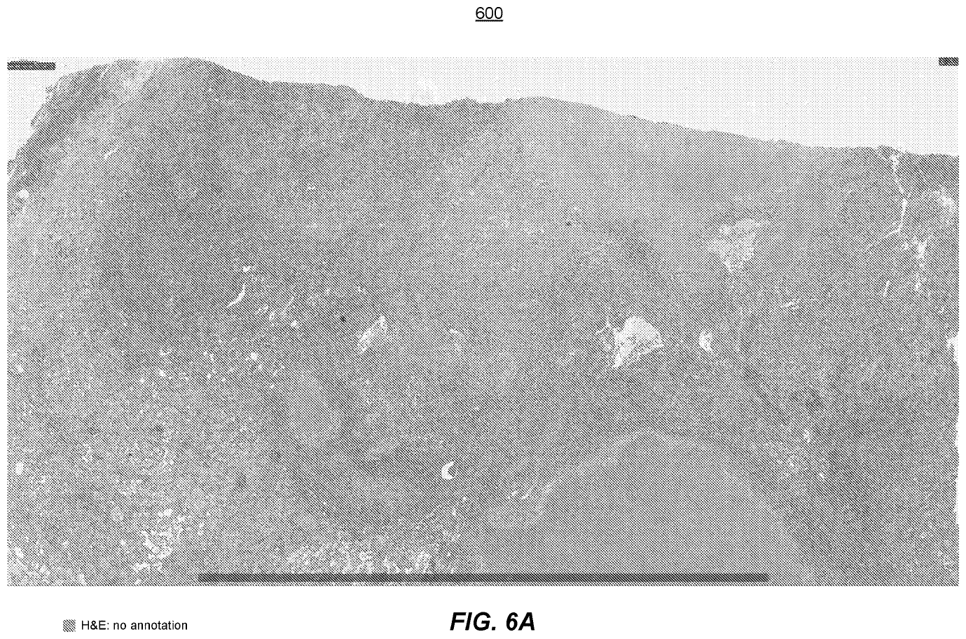

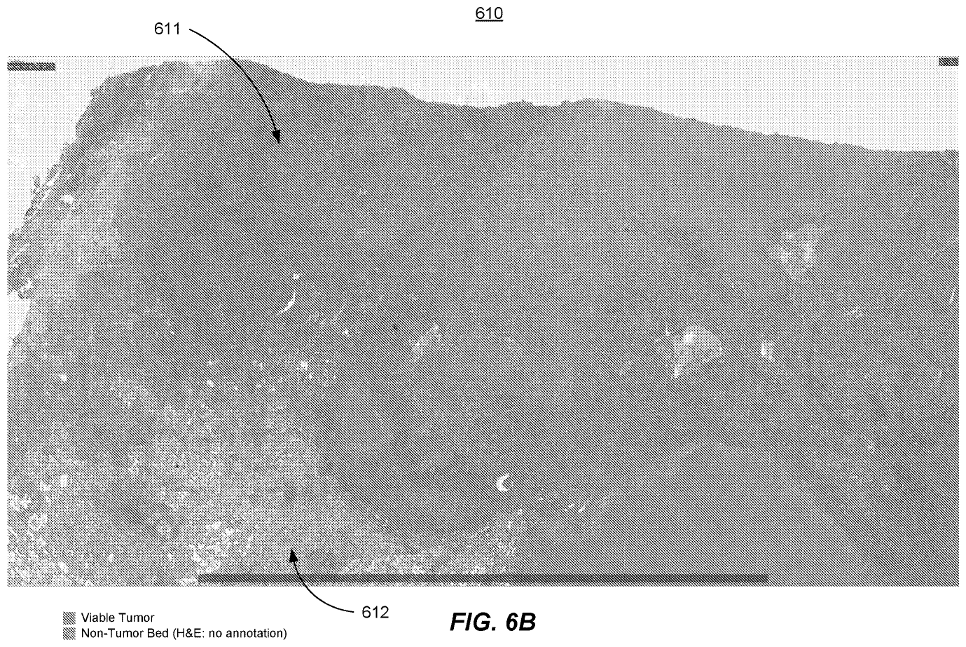

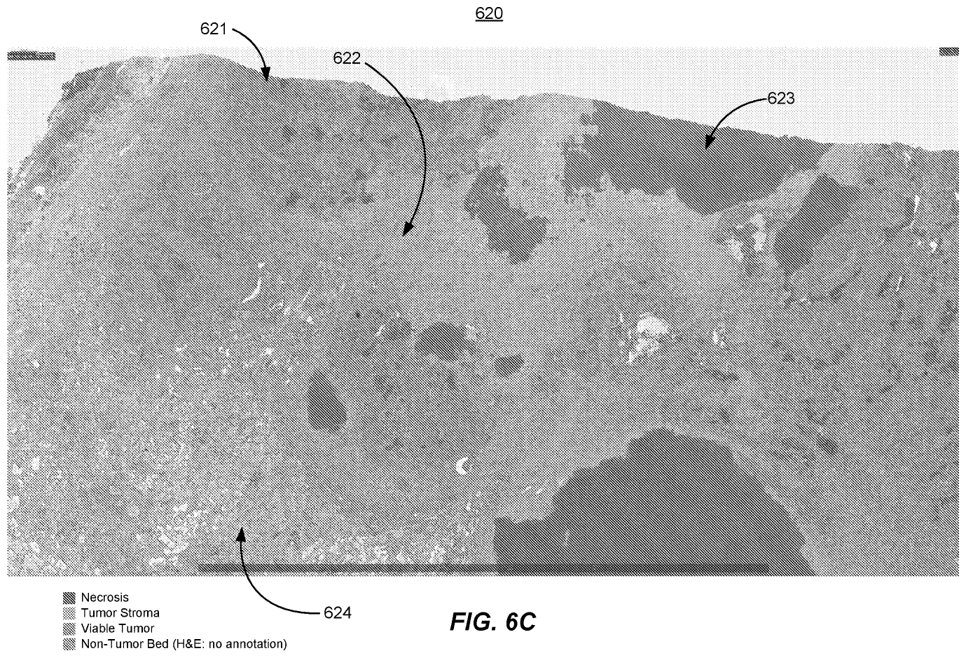

FIGS. 6A-8B illustrate examples of digital pathology images and output annotations produced by the digital pathology image processing system 120. FIGS. 6A-6C show three examples of digital pathology images 600, 610, and 620. As shown in FIG. 6A, example original digital pathology image 600 is a scan of an H&E-stained image (shown in typical purple and pink colors). Other stains, used independently or in combination, may also be used based on the trained models and the assessment goals of the digital pathology image processing system 120. As shown in FIG. 6B, example annotated image 610 represents digital pathology image 600 after evaluation by the tumor bed model 320. Annotated image 610 includes areas marked by the tumor bed model 320 to indicate that they correspond to the tumor bed (areas 611 shown in red) the portions of image 610 without any annotation (area 612 shown in purple and pink colors) illustrate adjacent non-tumor tissue. As shown in FIG. 6C, example annotated image 620 represents digital pathology image 600 after evaluation by the tissue model 325. The annotated image 620 includes sections marked by the tissue model 325 to indicate whether they correspond to viable tumor cells (various small areas 621 shown in red), tumor stroma (areas 622 shown in orange), or necrosis (various areas 623 shown in dark gray). The portions of the image without any annotation (areas 624 shown in pink and purple) illustrate adjacent non-tumor tissue.

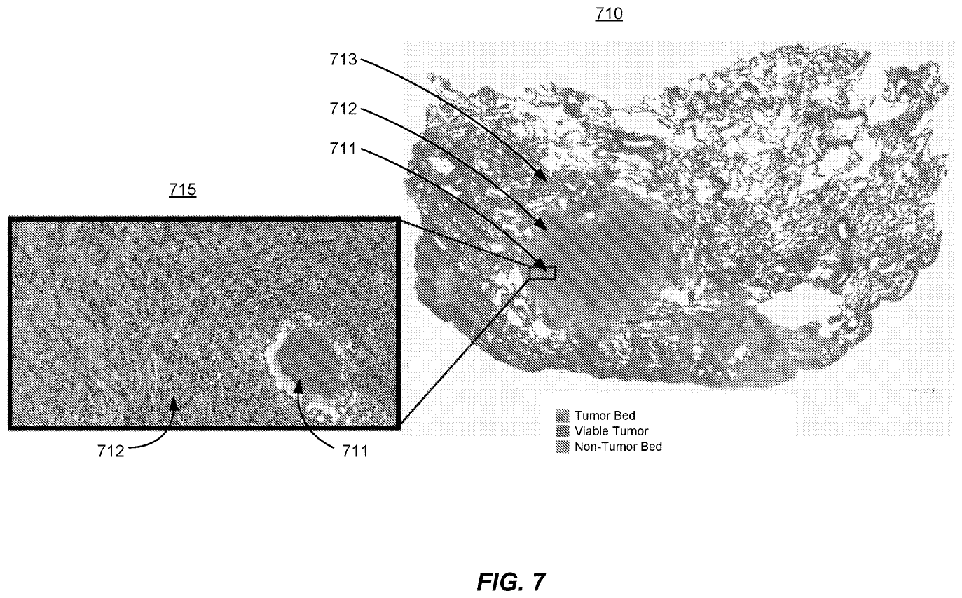

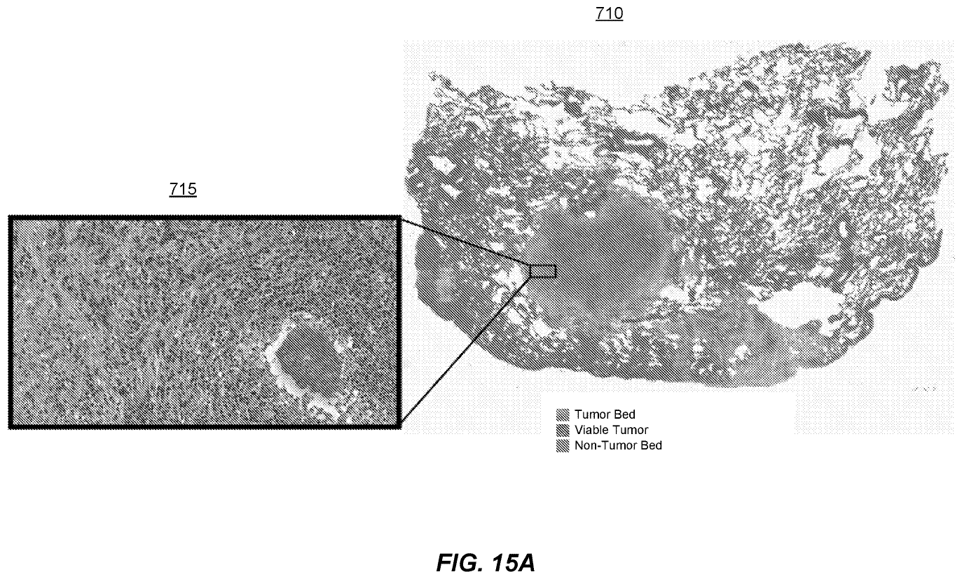

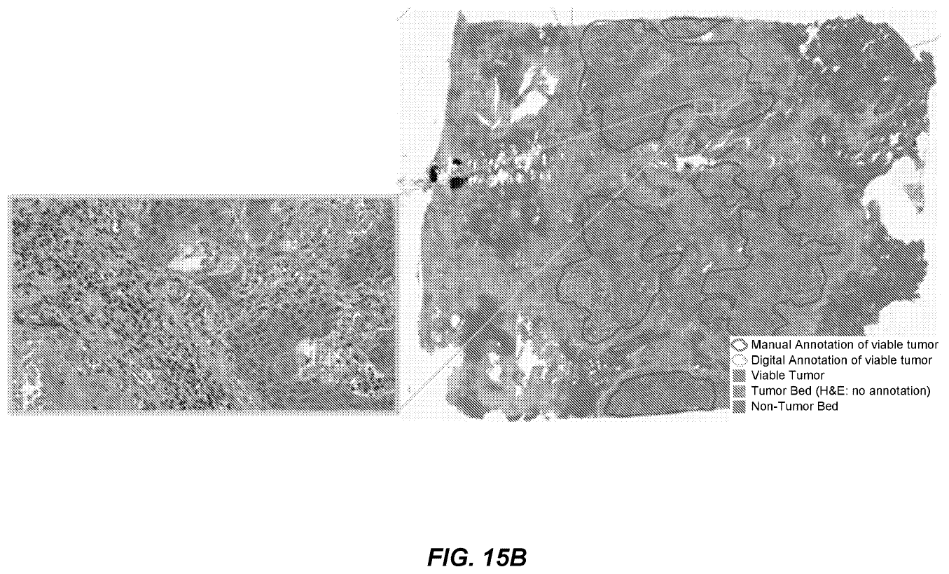

FIG. 7 includes an illustration of a digital pathology image 710 that has been evaluated by the tumor bed model 320. The annotated image 710 includes areas marked by the tumor bed model 320 to indicate that they correspond to viable tumor (areas 711 shown in red), tumor bed (areas 712 shown in purple), and adjacent non-tumor tissue (areas 713 shown in gray). FIG. 7 also illustrates an enhanced view (at higher magnification) of a region 715 of the image 710. Within the magnified enhanced view 715, annotations include annotations corresponding to residual viable tumor (areas 711 shown in red), and the portions of image 715 without any annotation (areas 712 shown in purple) illustrate adjacent inflammation within the tumor bed. This annotation style may be used to assist pathologists in reviewing images after assessment to help identify tumor cells of interest. FIG. 7 is discussed in further detail below as FIG. 15A.

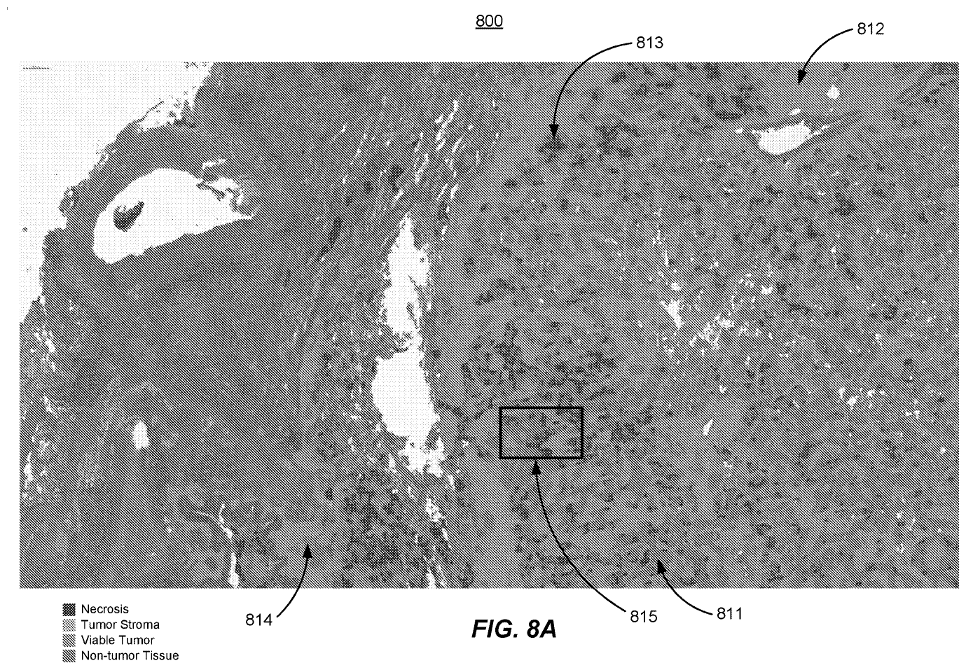

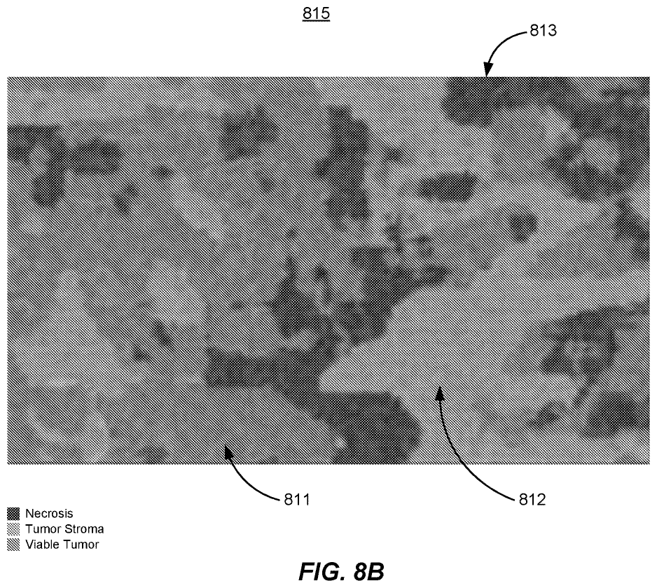

FIG. 8A includes another illustration of a digital pathology image 810 that has been evaluated by the tissue model 325. The annotated image 810 includes sections marked by the tissue model 325 to indicate whether they correspond to viable tumor (areas 811 shown in red), tumor stroma (areas 812 shown in orange), or necrosis (areas 813 shown in dark brown). The portions of the image without any annotation (areas 814 shown in purple) illustrate adjacent non-tumor tissue. Region 815 indicates an area shown at higher magnification in FIG. 8B. FIG. 8B includes an illustration of an enhanced view 815 of the image 810. Within the magnified enhanced view 815, annotations include annotations corresponding to viable tumor (areas 811 shown in red), tumor stroma (areas 812 shown in orange), and necrosis (areas 813 shown in dark brown).

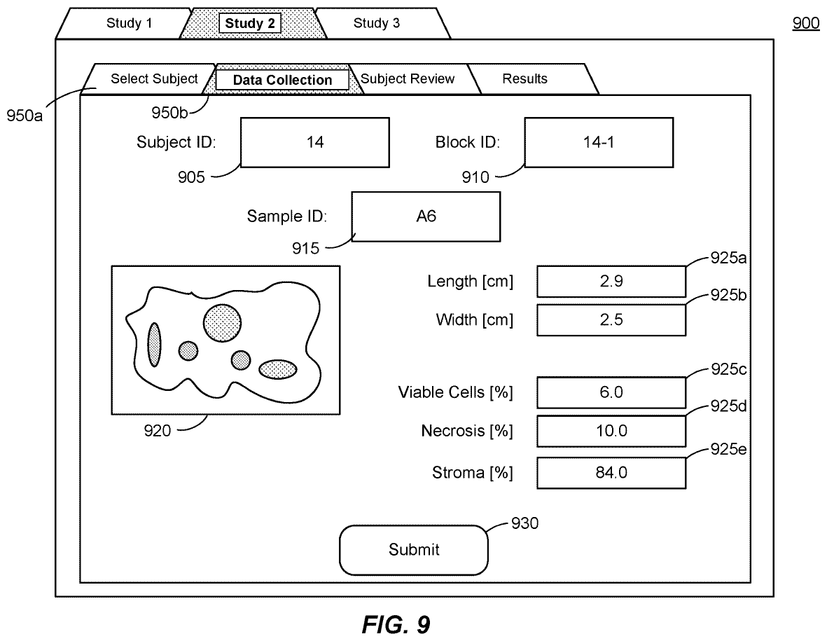

FIG. 9 illustrates an example interface 900 for the next stage of the Digital pathology image processing system 120 data collect workflow, corresponding to the “Sample Data Entry” tab 950b. The interface 900 may include a series of interactive fields enabling the operator to request and review key data for the sample needed for MPR evaluation and measured by the digital pathology image processing system 120. The example interface 900 illustrated in FIG. 9, includes the field 905 that shows the subject identifier and a field 910 that shows the block identifier, which were both entered in a “Select Subject” tab 950a. Interactive field 915 is a text field allowing the operator to enter a sample identifier for the sample for which they are entering or verifying the rest of the data to be entered. In some embodiments, the sample identifier may be assigned at an earlier time to correlate the information from a block with other uses. For example, where the block corresponds to a portion of resected tissue (e.g., of a tumor) the sample identifier may have been assigned by the surgeon who removed the tissue or by another technician. In other embodiments, the sample identifier may be assigned by the digital pathology image processing system 120 to ensure that unique values for each sample are entered.

The interface 900 may include an element 920 showing one or more images of the sample under evaluation by the digital pathology image processing system 120. As an example, where the sample is taken from a block of resected tissue, the image may be a digital image of a slide used to evaluate the tissue. As another example, where the sample is taken using medical imaging technology (e.g., a CT scan, PET scan, MRI, x-ray, etc.), the image 920 may be a digital image of said scan. The operator 920 may select the image 920 to zoom in on the image or view the image in a larger size or higher resolution (e.g., where the displayed image 920 is initial a thumbnail). In particular embodiments, the image 920 may be used by the operator prior to requesting the digital pathology image processing system 120 to evaluate the sample prior to verify that the data corresponds to the correct sample or to confirm that the data is appropriate for the sample. Interactive elements 925a-925e include various fields displaying the data corresponding to the sample indicated by the sample identifier 915. As an example, interactive field 925a allows the operator to review the length of the sample, interactive field 925b allows the operator to review the width of the field, interactive field 925c allows the operator to review the percentage of the area of the sample corresponding to viable cells, interactive field 925d allows the operator to review the percentage of the area of the sample corresponding to necrosis, and interactive field 925e allows the operator to review the percentage of the area of the sample corresponding to stroma. As discussed herein, the operator may modify the values generated by the digital pathology image processing system 120 to provide corrections or updated data for used by the digital pathology image processing system 120.

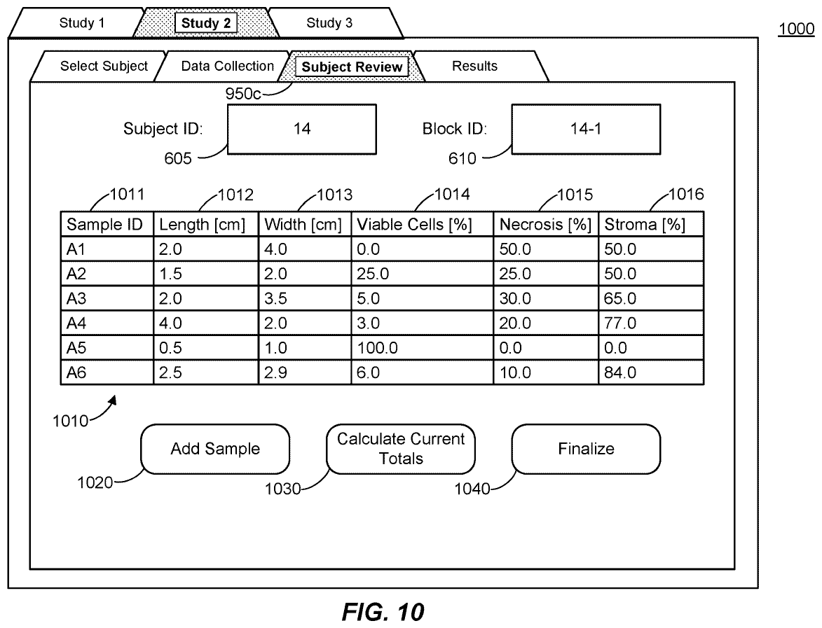

Once the user has reviewed or revised the values for this sample, interactive element 930 may be selected to save these results and submit them to the digital pathology image processing system 120. In particular embodiments, after submitting the information, the operator may be prompted whether they would like to add data for an additional sample to the current collection or whether they would like to review what has been and assessed by the digital pathology image processing system 120. In some embodiments, the interface 900 may include additional interactive elements to allow the user to specify whether they would like to review additional data or to review the all submitted data for the block without requiring an additional prompt. After the user has entered valid data and either automatically or manually advanced to the next stage of the digital pathology image processing system 120 workflow, the digital pathology image processing system 120 may display the interface 1000 illustrated in FIG. 10.

FIG. 10 illustrates an example interface 1000 corresponding to the “Subject Review” tab 950c for efficiently reviewing all entered data for the block. As with interface 900, the interface 1000 corresponding to the “Subject Review” tab 950c includes the field 905 that shows the subject identifier and a field 910 that shows the block identifier. The interface 1000 includes a table 1010 that displays all of the values entered for the samples submitted by the operator (e.g., requested by the operator of the digital pathology image processing system 120) for the block identified in by the displayed block identifier in field 910. The table 1010 as illustrated, includes a row for each submitted sample, a column 1011 for identifying the sample identifier, and columns 1012-1016 corresponding to the data collected for the various samples. In the example interface 1000 illustrated in FIG. 10, the columns include a column 1012 for sample length, a column 1013 for sample width, a column 1014 for the percentage of the area of the sample comprising viable cells, a column 1015 for the percentage of the area of the sample comprising necrosis, and a column 1016 for the percentage of the area of the sample comprising stroma. In particular embodiments, the operator may interact with a cell in the table to revise a value (e.g., to modify the length entered for a given sample). Additionally or alternatively, the operator may interact with a row to cause the digital pathology image processing system 120 to display the interface 900 corresponding to the “Sample Data Entry” tab 950b to review the sample (including the sample image) and potentially revise the submitted data.

The interface 1000 further includes a series of interactive elements providing additional functionality. Upon selecting interactive element 1020, the digital pathology image processing system 120 may transition to the interface 900 corresponding to the “Sample Data Entry” tab 950b for a new sample. Therefore, the interactive element 1020 may be used to submit additional samples data. Upon selecting the interactive element 1030, the digital pathology image processing system 120 may calculate running totals and/or averages for the data submitted for the identified block so far. The digital pathology image processing system 120 may display the calculated totals and averages in a new row of the table 1010, in a pop-up interactive element, or in another interface of the digital pathology image processing system 120. Upon selecting interactive element 1040, the digital pathology image processing system 120 may transition to the final stage of the sample data entry workflow for the digital pathology image processing system 120, where the operator may review the totality of the output produced by the digital pathology image processing system 120 and submit the final results to the study record.

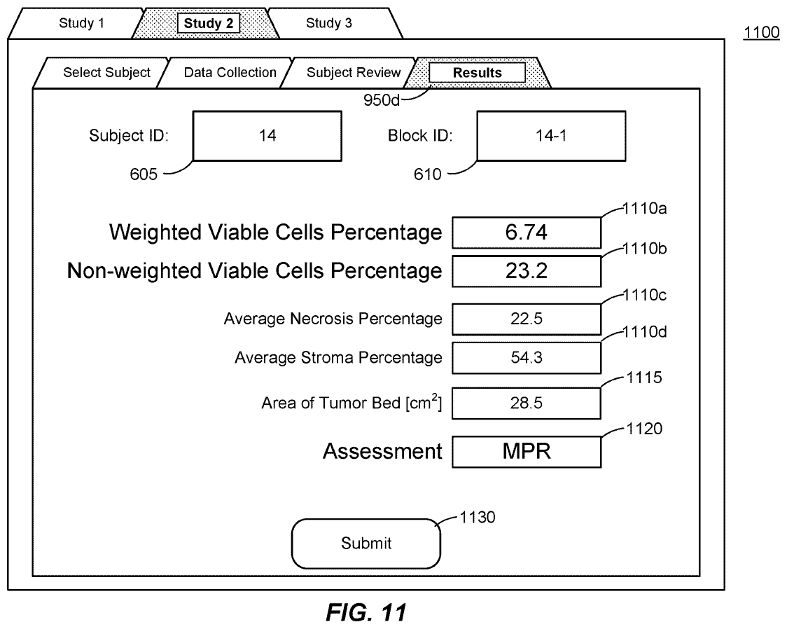

FIG. 11 illustrates an example interface 1100 for displaying calculated totals and reviewing MPR assessment results. The interface 1100 therefore corresponds to the “Results” tab 950d. As with interface 1100, the interface 1000 corresponding to the “Subject Review” tab 950c includes the field 1105 that shows the subject identifier and a field 1110 that shows the block identifier. The interface also displays the totals of interest for the block, which may be customized by the designer of the clinical study. As an example, the interface 1100 illustrated in FIG. 11 includes a field 110a to display the weighted percentage of the area of the samples submitted for the block that comprise viable cells, a field 1110b to display the non-weighted percentage of the samples submitted for the block that comprise viable cells, a field 1110c to display the average percentage of the samples submitted for the block that comprise necrosis, a field 1110d to display the average percentage of the samples submitted for the block that comprise stroma, and a field 1115 to display the total assessed area of the sample area. The values are all calculated by the digital pathology image processing system 120 based on the digital pathology images for the various samples as submitted by the operator. Additionally, the results reporting interface 1100 may include additional fields that may be customized for the particular study or operator. For example, the reporting interface 1100 may include fields to display the size of the sample area (e.g., tumor bed) at other points in time (e.g., pre-therapy, post-therapy), display an approximate percentage of the total mass examined, display the weighted and non-weighted percentages of other assessed or computed values (e.g., necrosis or stroma), etc.

The interface 1100 also includes a field 1120 that displays the assessment of the digital pathology image processing system 120. The field 1120 may include a simple yes or no determination for a particular type of result (e.g., whether MPR has been detected), which may enhance the usability of the digital pathology image processing system 120 (e.g., for diagnostic or evaluative purposes in addition to clinical studies). As another example, the field 1120 may include a determination and listing of whether one of a set of conditions have been detected (e.g., MPR, pCR, or other, etc.). As discussed herein, the condition being evaluated for and the assessment of MPR may be based on whether one or more of the calculated values (or a combination therefore) satisfies a threshold that may be set by the designer of the clinical study and which may be vary based on the type of samples being evaluated. The assessment performed may be determined by the digital pathology image processing system 120 itself based on other entered values. The interface 1100 also includes an interactive element 1130 for the operator to submit the final values to the study record. At any point before the operator submits the final values, the operator may easily move between the stages of the workflow by simply interacting with any of the set of tabs, which also indicate where in in the workflow the current interface being displayed is situated.

In addition to the interfaces discussed previously, the digital pathology image processing system 120 may comprise interfaces facilitating review of submit data by a second operator. As an example, the second operator may be another pathologist whose responsibility is to confirm that entered data is reasonable and correct. The second operator may be a study lead reviewing the data before it is compiled. The second operator may also be another data reviewer. To assist the second operator in reviewing the submitted data, the digital pathology image processing system 120 may include one or more interfaces directed to a prioritized review workflow that highlights and directs the second operator to data that the digital pathology image processing system 120 has flagged as requiring intervention from an operator by potentially including incorrect data, outliers, or other data of interest. The prioritized review workflow may be adaptive, directing the second operator review in a sequence of views based on a learned or crowd-sourced prioritization of detected anomalies or possible errors.

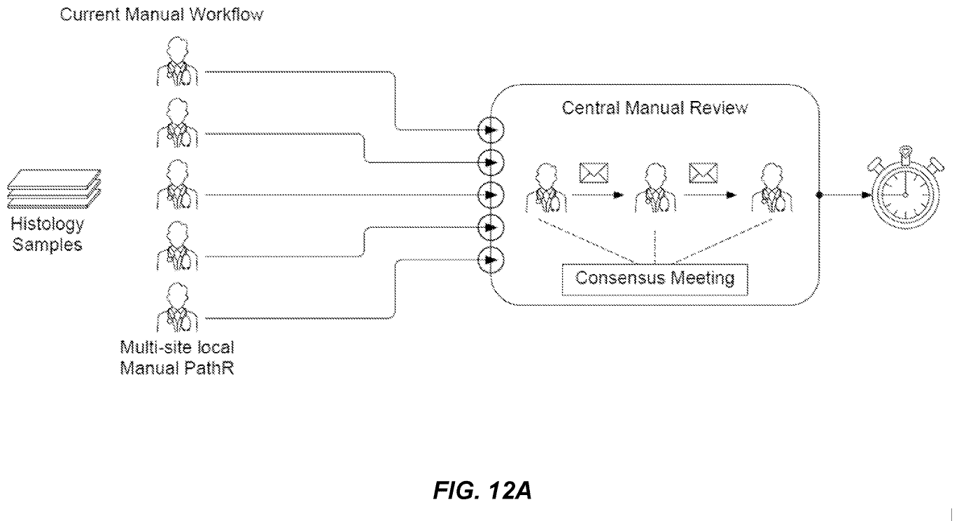

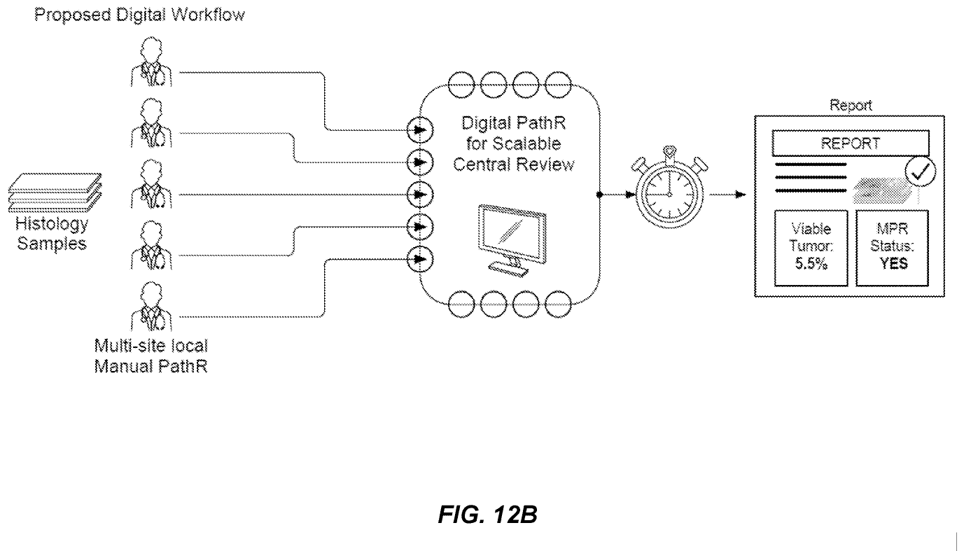

FIG. 12A illustrates a manual workflow for clinicians evaluating histology samples. FIG. 12B illustrates a digital workflow for automated assessment of pathologic response, according to embodiments of the present disclosure. Current workflows for evaluating histology samples in clinical trials involve evaluation for pathologic response of samples in multiple cases by one or more study-assigned pathologists at the local sites, followed by manual central review of the cases by one or more central reviewers to establish consensus. However, to date, there have been no investigations into the degree of variability from pathologist to pathologist. Automated machine-learning based approaches could enable scalable standardized quantification of tumor bed and residual viable tumor areas and may provide a complementary tool or an alternative to manual pathologic response assessment after neoadjuvant therapy to simplify clinical trial operations and eventually be used in clinical practice In this digital workflow, quantification calculations may be automated with pathologist supervision, so that the pathologist has more time to focus on complex or unusual cases that require manual interpretation.

The Phase II Lung Cancer Mutation Consortium 3 (LCMC3) study (NCT02927301) evaluated pre-operative atezolizumab (anti-PD-L1) in patients with untreated stage IB to IIIB resectable NSCLC (N=181) and achieved its primary endpoint with a 20% MPR rate. This trial provided an opportunity to explore the potential role of digital pathology in evaluating pathologic response and to correlate digitally assessed MPR with longer-term outcomes such as DFS. In LCMC3, inter-reader variability among pathologists was determined to evaluate agreement among local and central pathologists. Then an AI-powered convolutional neural network (CNN) model was developed to digitally assess percent viable tumor and MPR in line with IASLC recommendations, which included training the model to distinguish and quantify tissue components such as tumor bed, tumor bed with treatment response, viable invasive tumor, necrosis, and tumor-associated stroma, as well as to exclude adjacent non-tumor associated lung parenchyma. Using this model to assess pathologic response, the level of agreement with pathologic response was determined manually by expert pathologists, and the association of both manual and digital MPR assessment was evaluated with DFS outcomes.

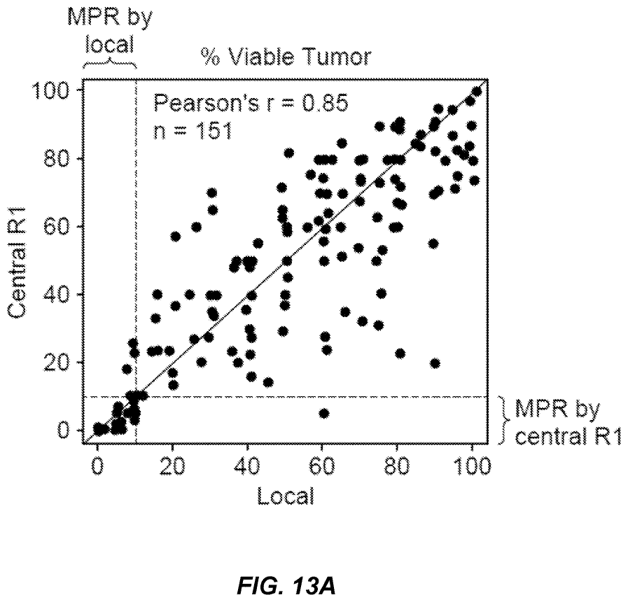

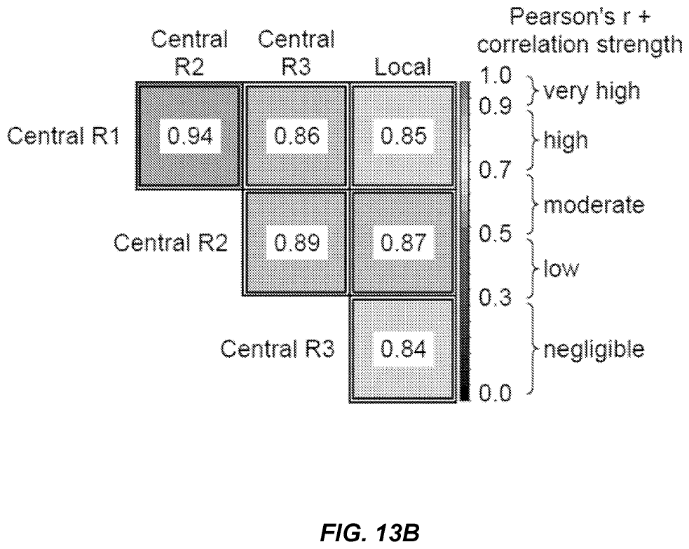

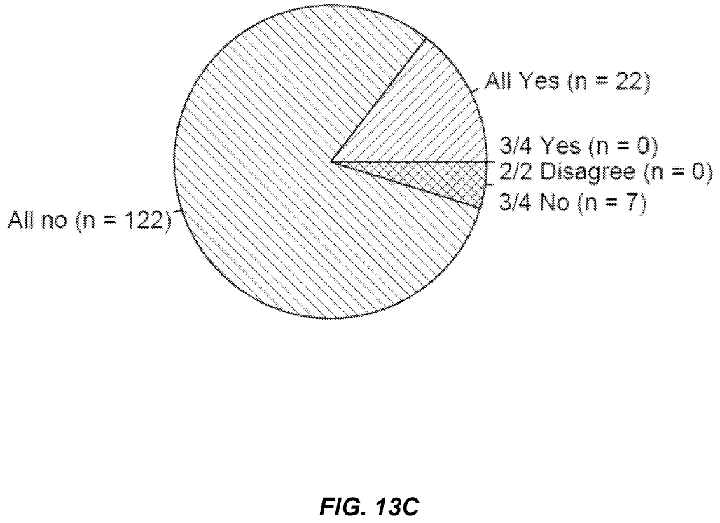

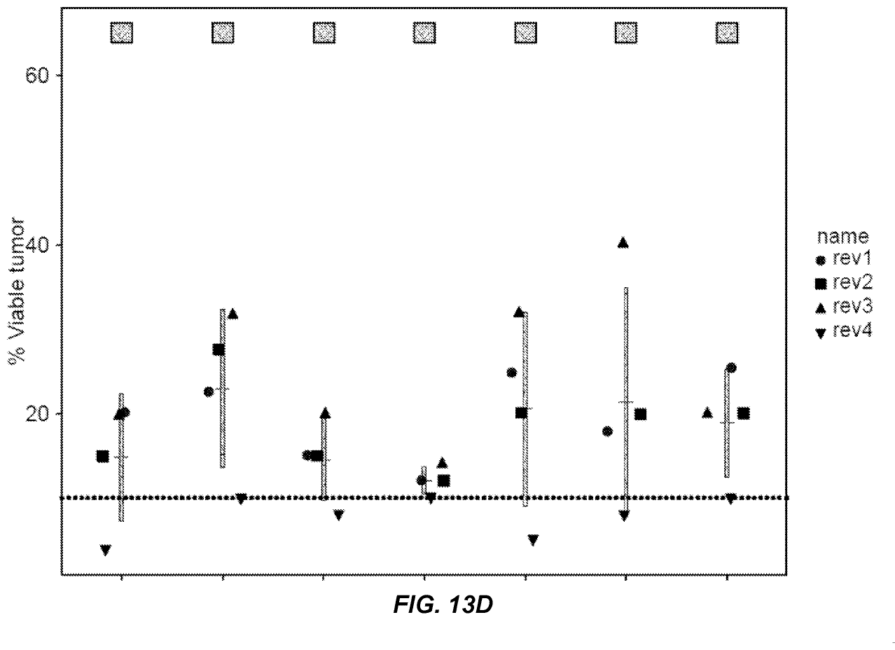

FIGS. 13A-13D illustrate various approaches to comparing manual assessment of percent viable tumor as between a local pathologist and three central reviewers Inter-reader agreement for manual assessment of percent viable tumor for 151 patients was evaluated among one local and three central pathologists. Substantial agreement was observed between local and central pathologists for manual assessment of percent viable tumor. As shown in FIG. 13A, Pearson's correlation between the local pathologist and one of the central reviewers was high to very high, as was the pairwise correlation between each of four readers (FIG. 13B). The intra-class correlation coefficient was 0.87 (95% CI: 0.84, 0.90) indicating good overall agreement.

Samples with 10% or less viable tumor cells were designated as achieving MPR. As shown in FIG. 13C, the percentage agreement for manual MPR among all four reviewers was 95.36 (Fleiss' kappa=0.9125633; 144 out of 151 cases in agreement). In all seven discordant cases, the local reviewer value tended to underestimate the percentage viable tumor as compared to the central reviewers (FIG. 13D).

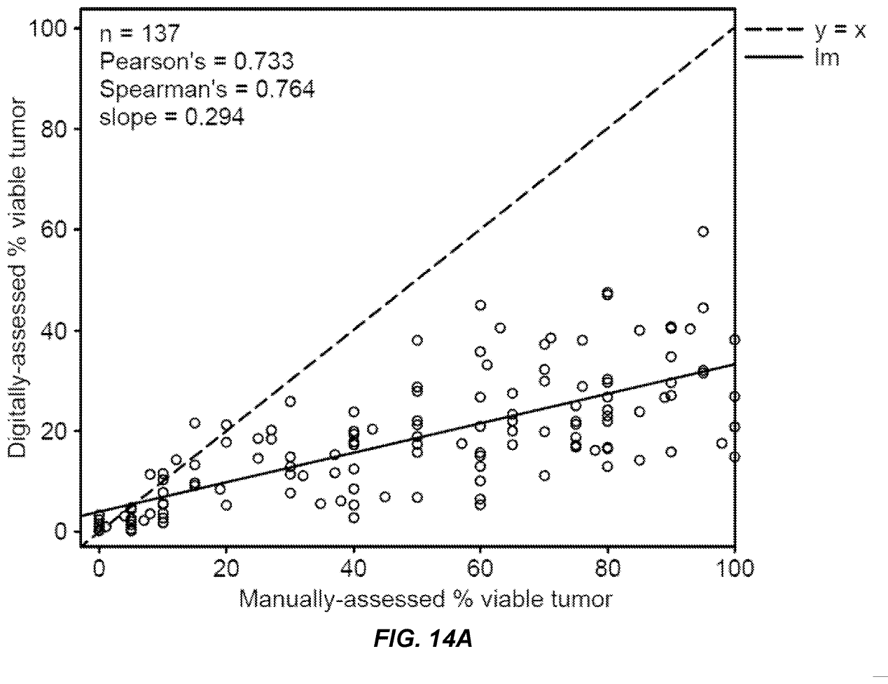

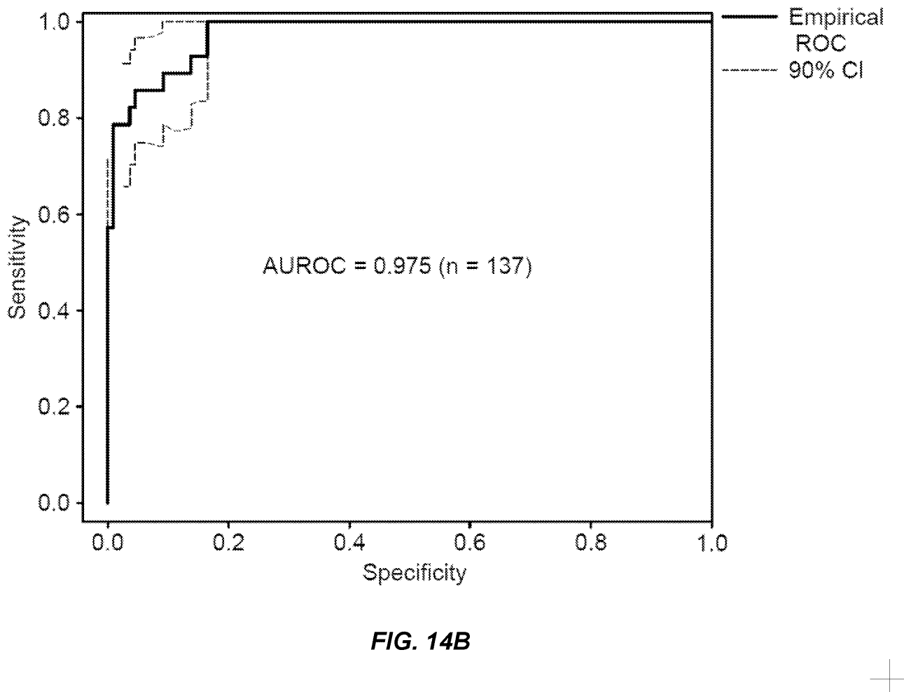

FIGS. 14A-14B illustrate example approaches to assessing performance of digital assessment of percent viable tumor by way of comparison to results obtained through manual assessment of percent viable tumor. Digital MPR shows outstanding ability to predict manual MPR. Histology samples from 137 patients were available to be digitized, with X images total (range 1-X per patient). Manually assessed percent viable tumor was strongly correlated with that obtained though digital assessment (Pearson's r=0.73; FIG. 14A), and digitally assessed MPR predicted manually assessed MPR with outstanding discrimination (AUROC=0.98; FIG. 14B). As shown in FIGS. 14A and 14B, y=0.284x+4.1773. The slope of the regression for digital as compared with manual percent viable tumor was 0.29 (FIG. 14A), indicating that the absolute percent viable tumor inferred digitally was often lower than the manually assessed value for a given sample.

FIGS. 15A-15B illustrate whole slide images depicting small regions of viable tumor. Concordance between digital and manual assessment appeared to be associated with differences in segmentation of regions. For example, viable tumor that appeared in distinct regions from tumor-associated stroma resulted in similar assessments. However, for clusters of viable tumor interlaced among tumor-associated stroma, the manual assessment of the percentage of viable tumor was numerically higher than for digital assessment. As shown in FIG. 15A (also shown in FIG. 7), small regions of viable tumor 711 were identified in tumor bed 712 (manual: 5% viable tumor; digital: 2.5% viable tumor). As shown in FIG. 15B, clusters of small regions of viable tumor in the tumor bed were grouped as a single region by manual assessment. Digital assessment parsed out the viable tumor that was interlaced among the tumor bed (manual: 40% viable tumor; digital: 20% viable tumor).

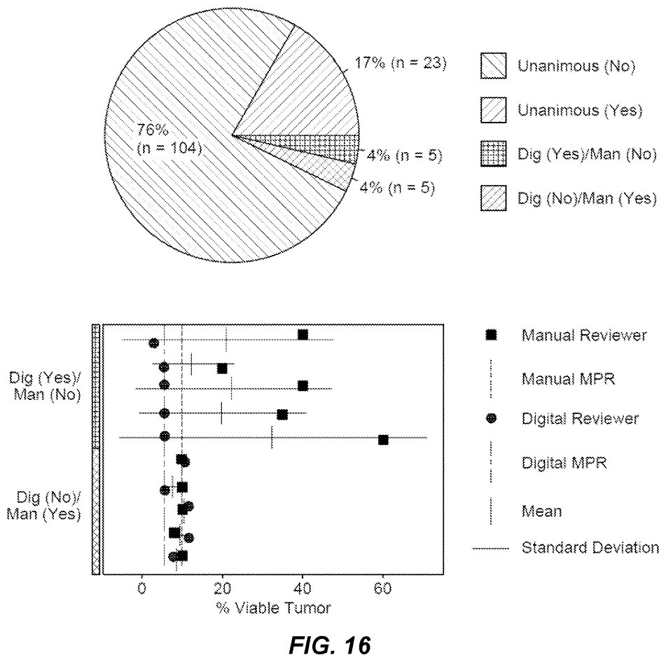

FIG. 16 shows two graphs comparing correlations and discrepancies between manual assessment of pathologic response and digital assessment of pathologic response. In order to adjust for the systematic differences in assessing percent viable tumor, a prevalence-matched MPR cutoff for digital assessment was determined by matching the prevalence of MPR cases to that of manual assessment. The resulting digital cutoff was 5.5% resulting in an agreement for determination of MPR of 93% (Cohen's kappa, 0.78). Based on this cutoff, digitally assessed MPR were discrepant with manual assessment in five MPR-yes cases and five MPR-no cases. Interestingly, the discrepant digital MPR-yes cases were all with discordant percent viable tumor, whereas the digital MPR-no cases had similar percent viable tumor values as the manually assessed values.

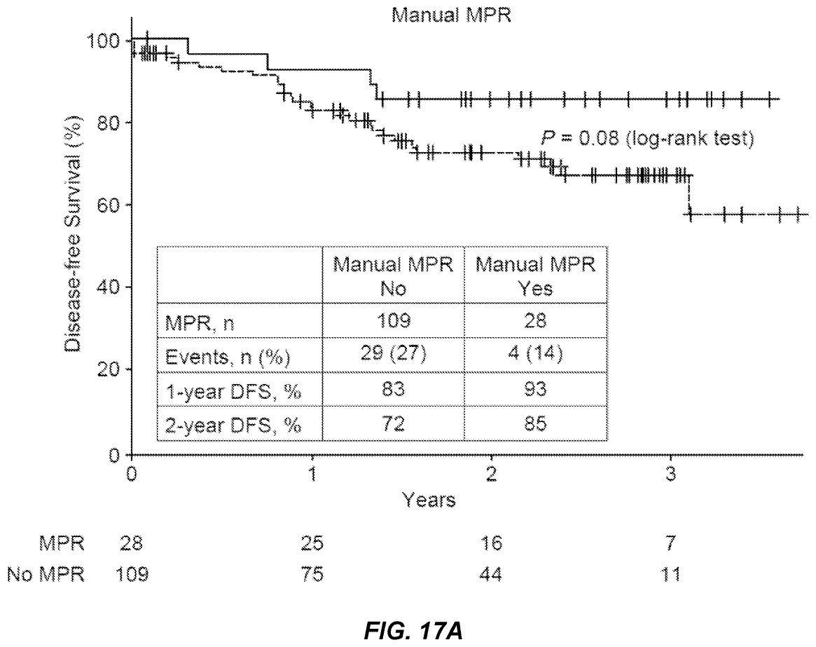

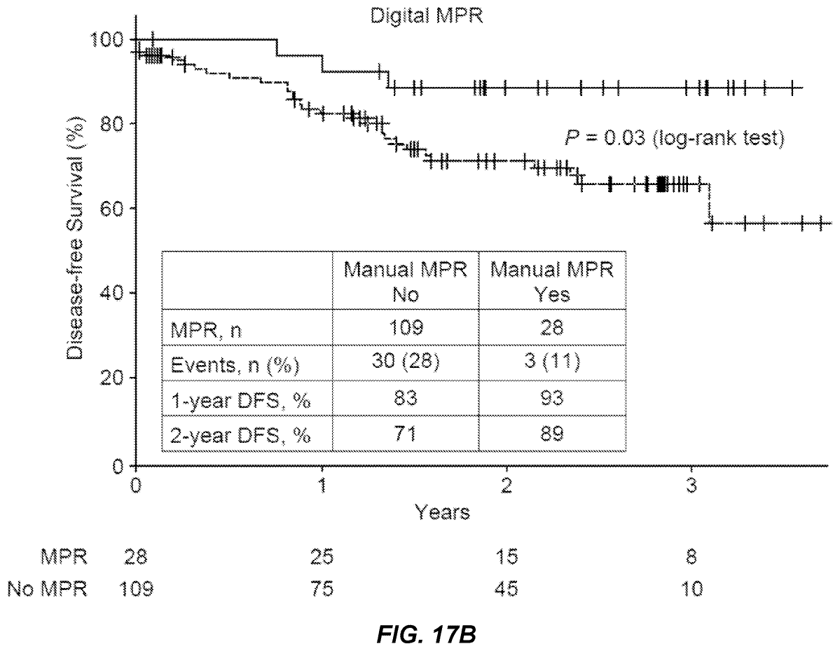

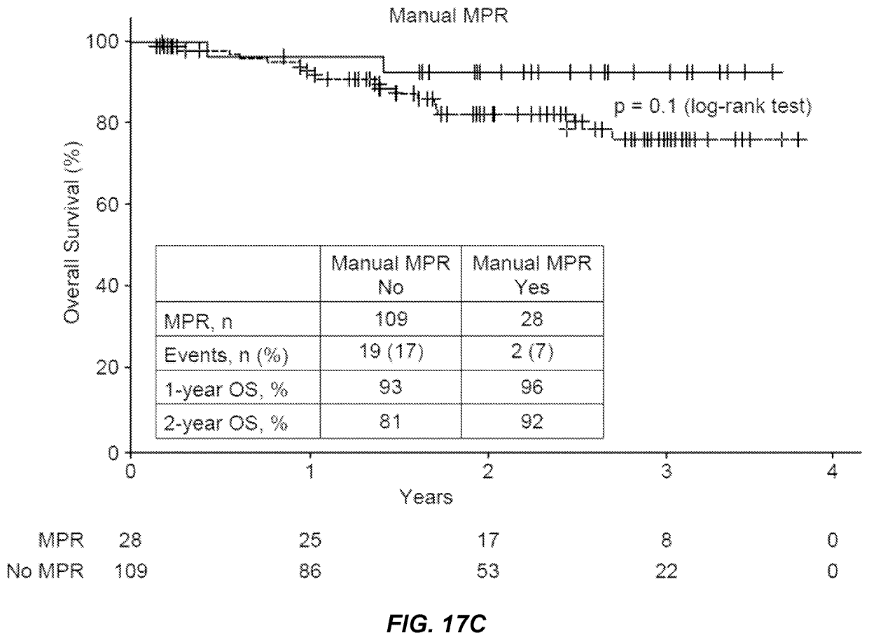

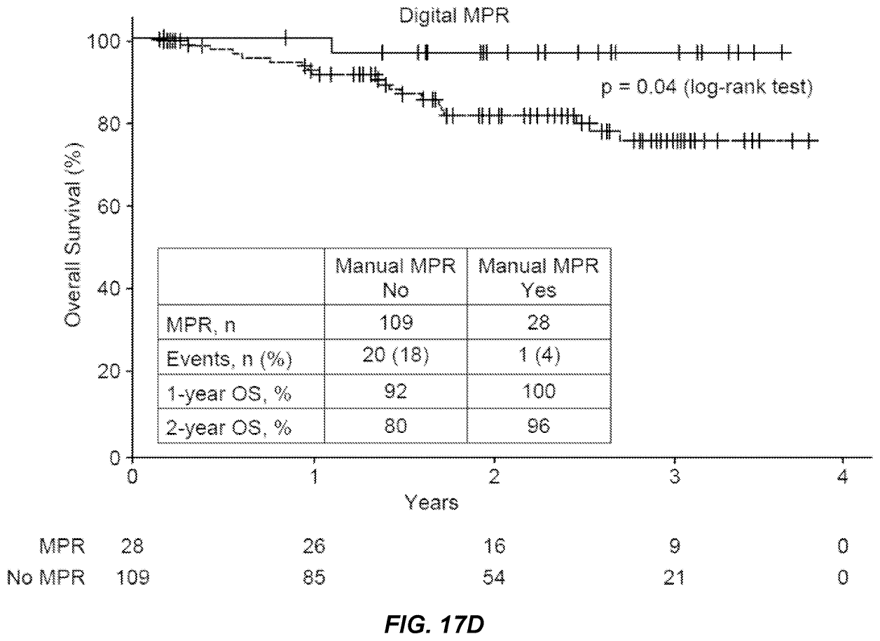

FIGS. 17A-17D illustrate four graphs showing differences between digitally assessed MPR versus manually assessed MPR with respect to DFS and OS. Disease-free survival (DFS) according to manually assessed MPR-yes showed a trend towards longer DFS vs. MPR-no that was not statistically significant (FIG. 17A). DFS according to digitally assessed MPR showed significantly longer DFS for MPR-yes vs. MPR-no (FIG. 17B). Similarly overall survival (OS) showed a non-significant trend towards longer OS for manually assessed MPR-yes vs. MPR-no (FIG. 17C) and a significantly longer OS for digitally assessed MPR-yes vs. MPR-no (FIG. 17D). When separating out cases based on their agreement between manual and digital MPR, being MPR-yes for both digital and manual shows the best long-term outcome. There is a trend toward all discordant cases doing worse than the perfectly concordant (MPR-yes for both manual and digital) cases, with a slight trend also for digital MPR-yes/manual MPR-no performing better than digital MPR-no/manual MPR-yes (but data is still immature, and the discordant case number is low).

Deep learning is being studied as a tool to assist in many areas of tumor pathology, including diagnosis; tumor subtyping, grading, or staging; evaluation of pathological features or biomarkers; and prognosis prediction. AI pathology has been used to predict survival benefit of adjuvant therapy in early-stage NSCLC. CNNs have also been used to identify and segment tumor areas on WSIs of lung tissue. The CNN was used primarily as a diagnostic tool, segmenting the tissue into areas of tumor and non-tumor.

This analysis showed that there was good inter-reader agreement for manual pathologic response in LCMC3 among local and central readers. There was also a strong correlation between AI-powered digital and manual pathologic response. Although there are limited studies on the reproducibility of pathologic response measurement, intra-class correlation coefficients (ICC) of 0.97 have been achieved between pathologists for assessing adenocarcinoma. Additionally, reproducibility is impacted by the magnitude of the true value, with values near 0% or 100% being more reproducible than those from 6% to 95% By optimizing training between the pathologists and the number of repeat assessments (median of 5) a Pearson R² of 0.994 (95% CI: 0.991, 0.996) was achieved, demonstrating that repeat measurements can lead to high reproducibility across the range of values. Thus, digital assessment could enhance accuracy by facilitating and accelerating repeat measurements at values where reproducibility is more challenging, particularly around the 10% cutoff for MPR.

By using a prevalence-matched cutoff for digital MPR, the DFS rates stratified by MPR-yes and -no were similar between manual and digital MPR in these preliminary results. These data support further studies of digital pathologic response as a standardized and scalable tool to determine pathologic response. Use of digital pathologic response may enable assessment of pathologic response to become more consistent across clinical trials and potentially across different clinical practices, such as academic and community settings. These results have demonstrated that digitally assessed pathologic response is a time-efficient and precise quantitative tool that pathologists can potentially adopt in a routine clinical practice.

However, while differences between local and central readers likely can be addressed through training or repeat measures, differences between manual and digital assessment may be subject to systematic methodological differences. While the formula for computing percent viable tumor is identical between manual and digital assessment methods, the model training and development process involved distinctions between tissue classes that differ from standard pathologist assessment. First, predictions at a pixel-scale provide extremely high resolution, as opposed to gross approximation of the size of regions of viable tumor. Second, pathologist-assessed viable tumor may include both cancer epithelium and nearby cancer-associated stroma that is subjectively determined to be part of the viable tumor, possibly due to assessment at low power. The high resolution of digitally assessed regions of viable tumor can provide a valuable tool to improve consistency of objective and quantitative evaluations.

A strength of this study is the large size of the training set and the training algorithm, which involved expert pathologists in each iterative round of training. A limitation of this study is the degree of training was less than some previous studies, resulting in lower ICCs than previously reported.

AI-powered digital pathology is being applied in ongoing correlative analyses in LCMC3 to investigate predictive and prognostic biomarkers and to further characterize the effect of neoadjuvant atezolizumab in the treatment of early-stage NSCLC. Further refinements in these AI models may eventually facilitate the optimization and personalization of treatment of these patients.

The assessment of major pathologic response relies on calculating the percent viable tumor, which is equal to the sum of the cancer epithelial area on all slides for a case, divided by the sum of the tumor bed area on all slides of the case. Three separate deep convolutional neural networks (CNNs) were developed and applied for pixel-wise classification of tissue type in each H&E histopathological whole-slide image (WSI) to replicate this calculation using digital pathology (see FIG. 4).

A first model (“artifact model”) was applied to identify tissue present on each slide, and predicted the presence of artifacts (e.g., tissue folding or blurring) within the WSI. Only regions deemed to contain usable tissue (i.e., not artifact or background) were used for subsequent modeling. One subsequent model (“tumor bed model”) was developed to classify tissue pixels from the artifact model as tumor bed or non-tumor bed. Another model (“tissue model”) was developed separately to classify tissue pixels from the artifact model as cancer epithelium, cancer-associated stroma, necrosis, or normal tissue. These three models were deployed to classify each pixel corresponding to tissue within each WSI as tumor bed or non-tumor bed, and one of cancer epithelium, stroma, necrosis, or normal tissue. The tumor-bed model predictions were then transformed to reconcile with the tissue model by reassigning every non-tumor bed pixel categorized as cancer epithelium, stroma, or necrosis as tumor bed.

To avoid overfitting on features from WSIs, machine-learning models were trained on a training dataset, evaluated and selected based on a validation dataset, and then deployed on a separate test set. The training dataset comprised 1984 WSIs from 1592 cases of proprietary and commercial NSCLC H&E-stained samples, of which 407 WSIs and 34 cases were obtained from the LCMC3 cohort. The validation dataset was reviewed during training and comprised an additional 292 WSIs from 205 cases, of which 97 WSI and 10 cases were from the LCMC3 cohort. Annotations by board-certified pathologists demarcating background, artifact, and tissue classifications on training and validation slides were collected. Models were trained and validated iteratively with a pathologist's evaluation included in each iteration. After each round of training, a board-certified pathologist reviewed the performance of the model by viewing the tissue classification predicted by the model overlaid onto an image of the WSI. Depending on the model's performance, additional annotations were collected for areas where the model performed poorly according to the pathologist. This process was repeated until qualitative and quantitative performance was deemed acceptable. In total 81,937 annotations of tumor bed, non-tumor bed, cancer epithelium, cancer-associated stroma, normal tissue, artifact, and background were collected and used for training and validation.

The models were then deployed retrospectively on the full dataset from the LCMC3 study, comprising 1671 WSIs from 154 resection cases. Model performance on the entire set was qualitatively assessed by pathologist review and comparison of pathologist-annotated regions to model predictions.

The outputs of these models, specifically, pixel-wise predictions of tumor bed and cancer epithelium classes, enabled replication of the manual viable tumor assessment by computing the case-wise digital percent viable tumor v^{d} as shown below:

$${v}^{d} = \frac{{\sum }_{i}^{N} {c}_{\dot{i}}^{d}}{{\sum }_{i}^{N} {b}_{i}^{d}} \times 100 \%$$

where cᵢ^{d} is the digitally assessed area of cancer epithelium and bᵢ^{d} is the digitally assessed area of tumor bed on slide i for each of N total H&E slides from the resection.

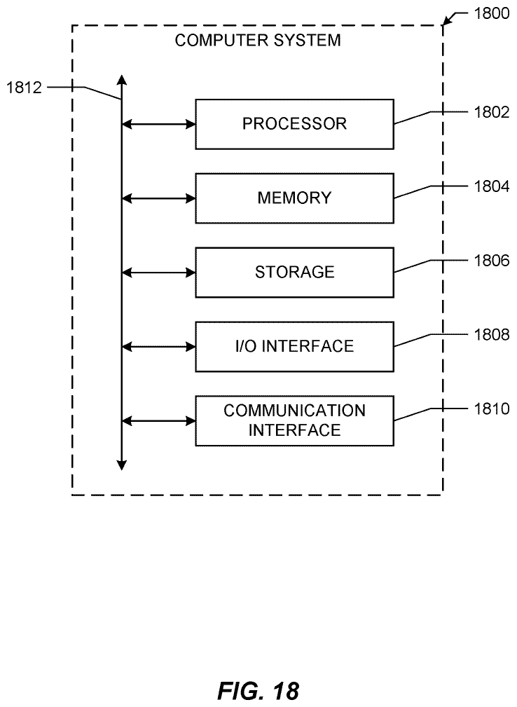

FIG. 18 illustrates an example computer system 1800. In particular embodiments, one or more computer systems 1800 perform one or more steps of one or more methods described or illustrated herein. In particular embodiments, one or more computer systems 1800 provide functionality described or illustrated herein. In particular embodiments, software running on one or more computer systems 1800 performs one or more steps of one or more methods described or illustrated herein or provides functionality described or illustrated herein. Particular embodiments include one or more portions of one or more computer systems 1800. Herein, reference to a computer system may encompass a computing device, and vice versa, where appropriate. Moreover, reference to a computer system may encompass one or more computer systems, where appropriate.

This disclosure contemplates any suitable number of computer systems 1800. This disclosure contemplates computer system 1800 taking any suitable physical form. As example and not by way of limitation, computer system 1800 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 1800 may include one or more computer systems 1800; be unitary or distributed; span multiple locations; span multiple machines; span multiple data centers; or reside in a cloud, which may include one or more cloud components in one or more networks. Where appropriate, one or more computer systems 1800 may perform without substantial spatial or temporal limitation one or more steps of one or more methods described or illustrated herein. As an example and not by way of limitation, one or more computer systems 1800 may perform in real time or in batch mode one or more steps of one or more methods described or illustrated herein. One or more computer systems 1800 may perform at different times or at different locations one or more steps of one or more methods described or illustrated herein, where appropriate.

In particular embodiments, computer system 1800 includes a processor 1802, memory 1804, storage 1806, an input/output (I/O) interface 1808, a communication interface 1810, and a bus 1812. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1802 includes hardware for executing instructions, such as those making up a computer program. As an example and not by way of limitation, to execute instructions, processor 1802 may retrieve (or fetch) the instructions from an internal register, an internal cache, memory 1804, or storage 1806; decode and execute them; and then write one or more results to an internal register, an internal cache, memory 1804, or storage 1806. In particular embodiments, processor 1802 may include one or more internal caches for data, instructions, or addresses. This disclosure contemplates processor 1802 including any suitable number of any suitable internal caches, where appropriate. As an example and not by way of limitation, processor 1802 may include one or more instruction caches, one or more data caches, and one or more translation lookaside buffers (TLBs). Instructions in the instruction caches may be copies of instructions in memory 1804 or storage 1806, and the instruction caches may speed up retrieval of those instructions by processor 1802. Data in the data caches may be copies of data in memory 1804 or storage 1806 for instructions executing at processor 1802 to operate on; the results of previous instructions executed at processor 1802 for access by subsequent instructions executing at processor 1802 or for writing to memory 1804 or storage 1806; or other suitable data. The data caches may speed up read or write operations by processor 1802. The TLBs may speed up virtual-address translation for processor 1802. In particular embodiments, processor 1802 may include one or more internal registers for data, instructions, or addresses. This disclosure contemplates processor 1802 including any suitable number of any suitable internal registers, where appropriate. Where appropriate, processor 1802 may include one or more arithmetic logic units (ALUs); be a multi-core processor; or include one or more processors 1802. Although this disclosure describes and illustrates a particular processor, this disclosure contemplates any suitable processor.

In particular embodiments, memory 1804 includes main memory for storing instructions for processor 1802 to execute or data for processor 1802 to operate on. As an example and not by way of limitation, computer system 1800 may load instructions from storage 1806 or another source (such as, for example, another computer system 1800) to memory 1804. Processor 1802 may then load the instructions from memory 1804 to an internal register or internal cache. To execute the instructions, processor 1802 may retrieve the instructions from the internal register or internal cache and decode them. During or after execution of the instructions, processor 1802 may write one or more results (which may be intermediate or final results) to the internal register or internal cache. Processor 1802 may then write one or more of those results to memory 1804. In particular embodiments, processor 1802 executes only instructions in one or more internal registers or internal caches or in memory 1804 (as opposed to storage 1806 or elsewhere) and operates only on data in one or more internal registers or internal caches or in memory 1804 (as opposed to storage 1806 or elsewhere). One or more memory buses (which may each include an address bus and a data bus) may couple processor 1802 to memory 1804. Bus 1812 may include one or more memory buses, as described below. In particular embodiments, one or more memory management units (MMUs) reside between processor 1802 and memory 1804 and facilitate accesses to memory 1804 requested by processor 1802. In particular embodiments, memory 1804 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1804 may include one or more memories 1804, where appropriate. Although this disclosure describes and illustrates particular memory, this disclosure contemplates any suitable memory.

In particular embodiments, storage 1806 includes mass storage for data or instructions. As an example and not by way of limitation, storage 1806 may include a hard disk drive (HDD), a floppy disk drive, flash memory, an optical disc, a magneto-optical disc, magnetic tape, or a Universal Serial Bus (USB) drive or a combination of two or more of these. Storage 1806 may include removable or non-removable (or fixed) media, where appropriate. Storage 1806 may be internal or external to computer system 1800, where appropriate. In particular embodiments, storage 1806 is non-volatile, solid-state memory. In particular embodiments, storage 1806 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1806 taking any suitable physical form. Storage 1806 may include one or more storage control units facilitating communication between processor 1802 and storage 1806, where appropriate. Where appropriate, storage 1806 may include one or more storages 1806. Although this disclosure describes and illustrates particular storage, this disclosure contemplates any suitable storage.

In particular embodiments, I/O interface 1808 includes hardware, software, or both, providing one or more interfaces for communication between computer system 1800 and one or more I/O devices. Computer system 1800 may include one or more of these I/O devices, where appropriate. One or more of these I/O devices may enable communication between a person and computer system 1800. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1808 for them. Where appropriate, I/O interface 1808 may include one or more device or software drivers enabling processor 1802 to drive one or more of these I/O devices. I/O interface 1808 may include one or more I/O interfaces 1808, where appropriate. Although this disclosure describes and illustrates a particular I/O interface, this disclosure contemplates any suitable I/O interface.

In particular embodiments, communication interface 1810 includes hardware, software, or both providing one or more interfaces for communication (such as, for example, packet-based communication) between computer system 1800 and one or more other computer systems 1800 or one or more networks. As an example and not by way of limitation, communication interface 1810 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1810 for it. As an example and not by way of limitation, computer system 1800 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1800 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1800 may include any suitable communication interface 1810 for any of these networks, where appropriate. Communication interface 1810 may include one or more communication interfaces 1810, where appropriate. Although this disclosure describes and illustrates a particular communication interface, this disclosure contemplates any suitable communication interface.

In particular embodiments, bus 1812 includes hardware, software, or both coupling components of computer system 1800 to each other. As an example and not by way of limitation, bus 1812 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1812 may include one or more buses 1812, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

### EMBODIMENTS

1. A one or more computer-readable non-transitory storage media comprising instructions executable by one or more processors of a digital pathology image processing system for:
receiving a plurality of digital pathology images of histologic samples;
assessing physical characteristics of a first histologic sample associated with a first digital pathology image of the plurality of digital pathology images;
segmenting the first digital pathology image based on one or more regions of the first digital pathology image corresponding to tumor bed;
segmenting the first digital pathology image based on one or more regions of the first digital pathology image corresponding to one or more predetermined histologic features;
generating an assessment regarding a specified condition in the first histologic sample based on the one or more regions of the first digital pathology image corresponding to tumor bed and the one or more regions of the first digital pathology image corresponding to the one or more predetermined histologic features; and
generating a user interface comprising a display of the assessment.

2. The one or more computer-readable non-transitory storage media of claim 1, wherein the segmenting the first digital pathology image based on one or more regions of the first digital pathology image corresponding to tumor bed is performed using a machine-learning model trained to segment tumor bed from non-tumor bed.

3. The one or more computer-readable non-transitory storage media of claims 1 or 2, wherein the segmenting the first digital pathology image based on one or more regions of the first digital pathology image corresponding to the one or more predetermined histologic features is performed using a machine-learning model trained to segment the one or more predetermined histologic features from tumor bed.

4. The one or more computer-readable non-transitory storage media of claim 3, further comprising instructions executable by one or more processors of the digital pathology image processing system for:
receiving, by the digital pathology image processing system, feedback from a user operator regarding the assessment; and
training the machine-learning model trained to segment the one or more predetermined histologic features from tumor bed based on the feedback.

5. The one or more computer-readable non-transitory storage media of any of claims 1 to 4, wherein the one or more predetermined histologic features include one or more of necrosis, viable tumor, and stroma.

6. The one or more computer-readable non-transitory storage media of any of claims 1 to 5, wherein generating the assessment regarding the specified condition comprises determining whether the specified condition is present.

7. The one or more computer-readable non-transitory storage media of any of claims 1 to 6, further comprising instructions executable by one or more processors of the digital pathology image processing system for:
computing a first area value of the first histologic sample corresponding to tumor bed based on the one or more regions of the first digital pathology image corresponding to tumor bed and the physical characteristics of the first histologic sample; and
computing a second area value of the first histologic sample corresponding to each of the one or more predetermined histologic features based on the one or more regions of the first digital pathology image corresponding to the one or more predetermined histologic features and the physical characteristics of the first histologic sample;
wherein the assessment regarding the specified condition in the first histologic sample is generated based on the first area value and the second area value.

8. The one or more computer-readable non-transitory storage media of claim 7, wherein determining whether the specified condition is detected in the first histologic sample based on the first area value and the second area value comprises computing a percentage of the second area relative to the first area corresponding to each of the one or more predetermined histologic features.

9. The one or more computer-readable non-transitory storage media of claim 8, wherein determining whether the specified condition is detected in the first histologic sample based on the first area value and the second area value comprises determining whether the percentage of the second area value relative to the first area value satisfies one or more predetermined thresholds, wherein the one or more predetermined thresholds are based on the specified condition.

10. The one or more computer-readable non-transitory storage media of any of claims 1 to 9, wherein:
segmenting the first digital pathology image based on the one or more regions of the first digital pathology image corresponding to tumor bed comprises producing a first instance of the first digital pathology image including annotations corresponding to the regions of the first digital pathology image corresponding to tumor bed; and
segmenting the first digital pathology image based on the one or more regions of the first digital pathology image corresponding to the one or more predetermined histologic features comprises producing a second instance of the first digital pathology image including annotations corresponding to the regions of the first digital pathology image corresponding to the one or more predetermined histologic features.

11. The one or more computer-readable non-transitory storage media of any of claims 1 to 10, wherein the first histologic sample is further associated with a set of one or more second digital pathology images;
wherein the one or more computer-readable non-transitory storage media further comprises, for each of the second digital pathology images:
   assessing physical characteristics of the first histologic sample associated with the second digital pathology image;
   segmenting the second digital pathology image based on one or more regions of the second digital pathology image corresponding to tumor bed; and
   segmenting the second digital pathology image based on one or more regions of the second digital pathology image corresponding to the one or more predetermined histologic features; and
wherein generating the assessment regarding the specified condition in the first histologic sample is further based on the one or more regions of the set of second digital pathology images corresponding to tumor bed and the one or more regions of the set of second digital pathology images corresponding to the one or more predetermined histologic features.

12. The one or more computer-readable non-transitory storage media of any of claims 1 to 11, further comprising instructions executable by one or more processors of the digital pathology image processing system for:
receiving a human-generated assessment of the first histologic sample;
comparing the assessment generated by the first digital pathology image processing system to the human-generated assessment; and
generating a user interface comprising a display of the comparison.

13. The one or more computer-readable non-transitory storage media of any of claims 1 to 12, wherein the assessment regarding the specified condition is further generated based on metadata and additional data associated with the first histologic sample.

14. The one or more computer-readable non-transitory storage media of any of claims 1 to 13, further comprising instructions executable by one or more processors of the digital pathology image processing system for generating a level of confidence in the assessment.

15. The one or more computer-readable non-transitory storage media of any of claims 1 to 14, wherein the user interface comprising the display of the assessment further comprises a display of annotations for the first digital pathology image associated with the segmentations based on the one or more regions of the first digital pathology image corresponding to tumor bed and the one or more regions of the first digital pathology image corresponding to the one or more predetermined histologic features.

16. A digital pathology image processing system comprising: one or more processors; and
one or more computer-readable non-transitory storage media coupled to one or more of the processors and comprising instructions operable when executed by one or more of the processors to cause the digital pathology image processing system to perform operations for:
receiving a plurality of digital pathology images of histologic samples;
assessing physical characteristics of a first histologic sample associated with a first digital pathology image of the plurality of digital pathology images;
segmenting the first digital pathology image based on one or more regions of the first digital pathology image corresponding to tumor bed;
segmenting the first digital pathology image based on one or more regions of the first digital pathology image corresponding to one or more predetermined histologic features;
generating an assessment regarding a specified condition in the first histologic sample based on the one or more regions of the first digital pathology image corresponding to tumor bed and the one or more regions of the first digital pathology image corresponding to die one or more predetermined histologic features; and
generating a user interface comprising a display of the assessment.

17. The digital pathology image processing system of claim 16 wherein the segmenting the first digital pathology image based on one or more regions of the first digital pathology image corresponding to tumor bed is performed using a machine-learning model trained to segment tumor bed from non-tumor bed.

18. The digital pathology image processing system of claims 16 or 17, wherein the segmenting the first digital pathology image based on one or more regions of the first digital pathology image corresponding to the one or more predetermined histologic features is performed using a machine-learning model trained to segment the one or more predetermined histologic features from tumor bed.

19. The digital pathology image processing system of claim 18, wherein the one or more computer-readable non-transitory storage media further comprise instructions operable when executed by one or more of the processors to cause the digital pathology image processing system to perform operations for:
receiving, by the digital pathology image processing system, feedback from a user operator regarding the assessment; and
training the machine-learning model trained to segment the one or more predetermined histologic features from tumor bed based on the feedback.

20. The digital pathology image processing system of any of claims 16 to 19, wherein the one or more predetermined histologic features include one or more of necrosis, viable cells, and stroma.

21. The digital pathology image processing system of any of claims 16 to 20, wherein generating the assessment regarding the specified condition comprises determining whether the specified condition is present.

22. The digital pathology image processing system of any of claims 16 to 21, wherein the one or more computer-readable non-transitory storage media further comprise instructions operable when executed by one or more of the processors to cause the digital pathology image processing system to perform operations for:
computing a first area value of the first histologic sample corresponding to tumor bed based on the one or more regions of the first digital pathology image corresponding to tumor bed and the physical characteristics of the first histologic sample;
computing a second area value of the first histologic sample corresponding to each of the one or more predetermined histologic features based on the one or more regions of the first digital pathology image corresponding to the one or more predetermined histologic features and the physical characteristics of the first histologic sample; and
wherein the assessment regarding the specified condition in the first histologic sample is generated based on the first area value and the second area value.

23. The digital pathology image processing system of claim 22, wherein determining whether the specified condition is detected in the first histologic sample based on the first area value and the second area value comprises computing a percentage of the second area relative to the first area corresponding to each of the one or more predetermined histologic features.

24. The digital pathology image processing system of claim 23, wherein determining whether the specified condition is detected in the first histologic sample based on the first area value and the second area value comprises determining whether the percentage of the second area value relative to the first area value satisfies one or more predetermined thresholds, wherein the one or more predetermined thresholds are based on the specified condition.

25. The digital pathology image processing system of any of claims 16 to 24, wherein:
segmenting the first digital pathology image based on the one or more regions of the first digital pathology image corresponding to tumor bed comprises producing a first instance of the first digital pathology image including annotations corresponding to the regions of the first digital pathology image corresponding to tumor bed, and
segmenting the first digital pathology image based on the one or more regions of the first digital pathology image corresponding to the one or more predetermined histologic features comprises producing a second instance of the first digital pathology image including annotations corresponding to the regions of the first digital pathology image corresponding to the one or more predetermined histologic features.

26. The digital pathology image processing system of any of claims 16 to 25, wherein the first histologic sample is further associated with a set of one or more second digital pathology images; and
wherein the one or more computer-readable non-transitory storage media further comprise instructions operable when executed by one or more of the processors to cause the digital pathology image processing system to perform operations, for each of the second digital pathology images, for:
   assessing physical characteristics of the first histologic sample associated with the second digital pathology image;
   segmenting the second digital pathology image based on one or more regions of the second digital pathology image corresponding to tumor bed; and
   segmenting the second digital pathology image based on one or more regions of the second digital pathology image corresponding to the one or more predetermined histologic features; and
wherein generating the assessment regarding the specified condition in the first histologic sample is further based on the one or more regions of the set of second digital pathology images corresponding to tumor bed and the one or more regions of the set of second digital pathology images corresponding to the one or more predetermined histologic features.

27. The digital pathology image processing system of any of claims 16 to 26, wherein the one or more computer-readable non-transitory storage media further comprise instructions operable when executed by one or more of the processors to cause the digital pathology image processing system to perform operations for.
receiving a human-generated assessment of the first histologic sample;
comparing the assessment generated by the first digital pathology image processing system to the human-generated assessment; and
generating a user interface comprising a display of the comparison.

28. The digital pathology image processing system of any of claims 16 to 27, wherein the assessment regarding the specified condition is further generated based on metadata and additional data associated with the first histologic sample.

29. The digital pathology image processing system of any of claims 16 to 28, wherein the one or more computer-readable non-transitory storage media further comprise instructions operable when executed by one or more of the processors to cause the digital pathology image processing system to perform operations for generating a level of confidence in the assessment.

30. The digital pathology image processing system of any of claims 16 to 29, wherein the user interface comprising the display of the assessment further comprises a display of annotations for the first digital pathology image associated with the segmentations based on the one or more regions of the first digital pathology image corresponding to tumor bed and the one or more regions of the first digital pathology image corresponding to the one or more predetermined histologic features.

31. A method comprising, by a whole slide image search system:
receiving a plurality of digital pathology images of histologic samples;
assessing physical characteristics of a first histologic sample associated with a first digital pathology image of the plurality of digital pathology images.
segmenting the first digital pathology image based on one or more regions of the first digital pathology image corresponding to tumor bed;
segmenting the first digital pathology image based on one or more regions of the first digital pathology image corresponding to one or more predetermined histologic features;
generating an assessment regarding a specified condition in the first histologic sample based on the one or more regions of the first digital pathology image corresponding to tumor bed and the one or more regions of the first digital pathology image corresponding to the one or more predetermined histologic features; and
generating a user interface comprising a display of the assessment.

32. The method of claim 31, wherein the segmenting the first digital pathology image based on one or more regions of the first digital pathology image corresponding to tumor bed is performed using a machine-learning model trained to segment tumor bed from non-tumor bed.

33. The method of claims 31 or 32, wherein the segmenting the first digital pathology image based on one or more regions of the first digital pathology image corresponding to the one or more predetermined histologic features is performed using a machine-learning model trained to segment the one or more predetermined histologic features from tumor bed.

34. The method of claim 33, further comprising:
receiving, by the digital pathology image processing system, feedback from a user operator regarding the assessment; and
training the machine-learning model trained to segment the one or more predetermined histologic features from tumor bed based on the feedback.

35. The method of any of claims 31 to 34, wherein the one or more predetermined histologic features include one or more of necrosis, viable cells, and stroma.

36. The method of any of claims 31 to 35, wherein generating the assessment regarding the specified condition comprises determining whether the specified condition is present.

37. The method of any of claims 31 to 36, further comprising:
computing a first area value of the first histologic sample corresponding to tumor bed based on the one or more regions of the first digital pathology image corresponding to tumor bed and the physical characteristics of the first histologic sample;
computing a second area value of the first histologic sample corresponding to each of the one or more predetermined histologic features based on the one or more regions of the first digital pathology image corresponding to the one or more predetermined histologic features and the physical characteristics of the first histologic sample; and
wherein the assessment regarding the specified condition in the first histologic sample is generated based on the first area value and the second area value.

38. The method of claim 37, wherein determining whether the specified condition is detected in the first histologic sample based on the first area value and the second area value comprises computing a percentage of the second area relative to the first area corresponding to each of the one or more predetermined histologic features.

39. The method of claim 38, wherein determining whether the specified condition is detected in the first histologic sample based on the first area value and the second area value comprises determining whether the percentage of the second area value relative to the first area value satisfies one or more predetermined thresholds, wherein the one or more predetermined thresholds are based on the specified condition.

40. The method of any of claims 31 to 39, wherein:
segmenting the first digital pathology image based on the one or more regions of the first digital pathology image corresponding to tumor bed comprises producing a first instance of the first digital pathology image including annotations corresponding to the regions of the first digital pathology image corresponding to tumor bed; and
segmenting the first digital pathology image based on the one or more regions of the first digital pathology image corresponding to the one or more predetermined histologic features comprises producing a second instance of the first digital pathology image including annotations corresponding to the regions of the first digital pathology image corresponding to the one or more predetermined histologic features.

41 The method of any of claims 31 to 40, wherein the first histologic sample is further associated with a set of one or more second digital pathology images;
wherein the one or more computer-readable non-transitory storage media further comprises, for each of the second digital pathology images:
   assessing physical characteristics of the first histologic sample associated with the second digital pathology image;
   segmenting the second digital pathology image based on one or more regions of the second digital pathology image corresponding to tumor bed; and
   segmenting the second digital pathology image based on one or more regions of the second digital pathology image corresponding to the one or more predetermined histologic features, and
wherein generating the assessment regarding the specified condition in the first histologic sample is further based on the one or more regions of the set of second digital pathology images corresponding to tumor bed and the one or more regions of the set of second digital pathology images corresponding to the one or more predetermined histologic features.

42. The method of any of claims 31 to 41, further comprising:
receiving a human-generated assessment of the first histologic sample;
comparing the assessment generated by the first digital pathology image processing system to the human-generated assessment; and
generating a user interface comprising a display of the comparison.

43. The method of any of claims 31 to 42, wherein the assessment regarding the specified condition is further generated based on metadata and additional data associated with the first histologic sample.

44. The method of any of claims 31 to 43, further comprising generating a level of confidence in the assessment.

45. The method of any of claims 31 to 44, wherein the user interface comprising the display of the assessment further comprises a display of annotations for the first digital pathology image associated with the segmentations based on the one or more regions of the first digital pathology image corresponding to tumor bed and the one or more regions of the first digital pathology image corresponding to the one or more predetermined histologic features.

What is claimed is:

1. A one or more computer-readable non-transitory storage media comprising instructions executable by one or more processors of a digital pathology image processing system for:
receiving a plurality of digital pathology images of histologic samples;
assessing physical characteristics of a first histologic sample associated with a first digital pathology image of the plurality of digital pathology images;
segmenting the first digital pathology image based on one or more regions of the first digital pathology image corresponding to tumor bed;
segmenting the first digital pathology image based on one or more regions of the first digital pathology image corresponding to one or more predetermined histologic features;
generating an assessment regarding a specified condition in the first histologic sample based on the one or more regions of the first digital pathology image corresponding to tumor bed and the one or more regions of the first digital pathology image corresponding to the one or more predetermined histologic features; and
generating a user interface comprising a display of the assessment.

2. The one or more computer-readable non-transitory storage media of claim 1, wherein the segmenting the first digital pathology image based on one or more regions of the first digital pathology image corresponding to tumor bed is performed using a machine-learning model trained to segment tumor bed from non-tumor bed.

3. The one or more computer-readable non-transitory storage media of claim 1, wherein the segmenting the first digital pathology image based on one or more regions of the first digital pathology image corresponding to the one or more predetermined histologic features is performed using a machine-learning model trained to segment the one or more predetermined histologic features from tumor bed.

4. The one or more computer-readable non-transitory storage media of claim 3, further comprising instructions executable by one or more processors of the digital pathology image processing system for:
receiving, by the digital pathology image processing system, feedback from a user operator regarding the assessment; and
training the machine-learning model trained to segment the one or more predetermined histologic features from tumor bed based on the feedback.

5. The one or more computer-readable non-transitory storage media of claim 1, wherein the one or more predetermined histologic features comprise necrotic tumor cells, regions of necrosis, viable tumor cells, regions of viable tumor, tumor stroma cells, or regions of tumor stroma.

6. The one or more computer-readable non-transitory storage media of claim 1, wherein generating the assessment regarding the specified condition comprises determining whether the specified condition is present.

7. The one or more computer-readable non-transitory storage media of claim 1, further comprising instructions executable by one or more processors of the digital pathology image processing system for:
computing a first area value of the first histologic sample corresponding to tumor bed based on the one or more regions of the first digital pathology image corresponding to tumor bed and the physical characteristics of the first histologic sample; and
computing a second area value of the first histologic sample corresponding to each of the one or more predetermined histologic features based on the one or more regions of the first digital pathology image corresponding to the one or more predetermined histologic features and the physical characteristics of the first histologic sample;
wherein the assessment regarding the specified condition in the first histologic sample is generated based on the first area value and the second area value.

8. The one or more computer-readable non-transitory storage media of claim 7, wherein determining whether the specified condition is detected in the first histologic sample based on the first area value and the second area value comprises computing a percentage of the second area relative to the first area corresponding to each of the one or more predetermined histologic features.

9. The one or more computer-readable non-transitory storage media of claim 8, wherein determining whether the specified condition is detected in the first histologic sample based on the first area value and the second area value comprises determining whether the percentage of the second area value relative to the first area value satisfies one or more predetermined thresholds, wherein the one or more predetermined thresholds are based on the specified condition.

10. The one or more computer-readable non-transitory storage media of claim 1, wherein:
segmenting the first digital pathology image based on the one or more regions of the first digital pathology image corresponding to tumor bed comprises producing a first instance of the first digital pathology image including annotations corresponding to the regions of the first digital pathology image corresponding to tumor bed; and
segmenting the first digital pathology image based on the one or more regions of the first digital pathology image corresponding to the one or more predetermined histologic features comprises producing a second instance of the first digital pathology image including annotations corresponding to the regions of the first digital pathology image corresponding to the one or more predetermined histologic features.

11. The one or more computer-readable non-transitory storage media of claim 1, wherein the first histologic sample is further associated with a set of one or more second digital pathology images;
wherein the one or more computer-readable non-transitory storage media further comprising instructions executable by one or more processors of the digital pathology image processing system for, for each of the second digital pathology images:
assessing physical characteristics of the first histologic sample associated with the second digital pathology image;
segmenting the second digital pathology image based on one or more regions of the second digital pathology image corresponding to tumor bed; and
segmenting the second digital pathology image based on one or more regions of the second digital pathology image corresponding to the one or more predetermined histologic features;
wherein generating the assessment regarding the specified condition in the first histologic sample is further based on the one or more regions of the set of second digital pathology images corresponding to tumor bed and the one or more regions of the set of second digital pathology images corresponding to the one or more predetermined histologic features.

12. The one or more computer-readable non-transitory storage media of claim 1, further comprising instructions executable by one or more processors of the digital pathology image processing system for:
receiving a human-generated assessment of the first histologic sample;
comparing the assessment generated by the first digital pathology image processing system to the human-generated assessment; and
generating a user interface comprising a display of the comparison.

13. The one or more computer-readable non-transitory storage media of claim 1, wherein the assessment regarding the specified condition is further generated based on metadata and additional data associated with the first histologic sample.

14. The one or more computer-readable non-transitory storage media of claim 1, further comprising instructions executable by one or more processors of the digital pathology image processing system for:
generating a level of confidence in the assessment.

15. The one or more computer-readable non-transitory storage media of claim 1, wherein the user interface comprising the display of the assessment further comprises a display of annotations for the first digital pathology image associated with the segmentations based on the one or more regions of the first digital pathology image corresponding to tumor bed and the one or more regions of the first digital pathology image corresponding to the one or more predetermined histologic features.

16. A digital pathology image processing system comprising: one or more processors; and
one or more computer-readable non-transitory storage media coupled to one or more of the processors and comprising instructions operable when executed by one or more of the processors to cause the digital pathology image processing system to perform operations comprising:
receiving a plurality of digital pathology images of histologic samples,
assessing physical characteristics of a first histologic sample associated with a first digital pathology image of the plurality of digital pathology images;
segmenting the first digital pathology image based on one or more regions of the first digital pathology image corresponding to tumor bed;
segmenting the first digital pathology image based on one or more regions of the first digital pathology image corresponding to one or more predetermined histologic features;
generating an assessment regarding a specified condition in the first histologic sample based on the one or more regions of the first digital pathology image corresponding to tumor bed and the one or more regions of the first digital pathology image corresponding to the one or more predetermined histologic features; and
generating a user interface comprising a display of the assessment.

17. The digital pathology image processing system of claim 16 wherein the segmenting the first digital pathology image based on one or more regions of the first digital pathology image corresponding to tumor bed is performed using a machine-learning model trained to segment tumor bed from non-tumor bed.

18. The digital pathology image processing system of claim 16, wherein the segmenting the first digital pathology image based on one or more regions of the first digital pathology image corresponding to the one or more predetermined histologic features is performed using a machine-learning model trained to segment the one or more predetermined histologic features from tumor bed.

19. A method comprising, by a digital pathology image processing system:
receiving a plurality of digital pathology images of histologic samples;
assessing physical characteristics of a first histologic sample associated with a first digital pathology image of the plurality of digital pathology images;
segmenting the first digital pathology image based on one or more regions of the first digital pathology image corresponding to tumor bed;
segmenting the first digital pathology image based on one or more regions of the first digital pathology image corresponding to one or more predetermined histologic features;
generating an assessment regarding a specified condition in the first histologic sample based on the one or more regions of the first digital pathology image corresponding to tumor bed and the one or more regions of the first digital pathology image corresponding to the one or more predetermined histologic features; and
generating a user interface comprising a display of the assessment.

20. The method of claim 19, wherein the segmenting the first digital pathology image based on one or more regions of the first digital pathology image corresponding to tumor bed is performed using a machine-learning model trained to segment tumor bed from non-tumor bed.
